SEQUENCE LISTING (82 sequences)

SEQ ID NO: 1 - protein, 98 aa, homo sapiens
QVQLVQSGAEVKKPGASVKVSCKASGYTFTGYYMHWVRQAPGQGLEWMGRINPNSGGTNYAQKFQGRVTSTRDTSISTAYMELSRLRSDDTVVYYCAR

SEQ ID NO: 2 - protein, 98 aa, homo sapiens
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCAR

SEQ ID NO: 3 - protein, 98 aa, homo sapiens
QVQLVQSGAEVKKPGASVKVSCKASGYTFTSYDINWVRQATGQGLEWMGWMNPNSGNTGYAQKFQGRVTMTRNTSISTAYMELSSLRSEDTAVYYCAR

SEQ ID NO: 4 - protein, 98 aa, homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 5 - protein, 98 aa, homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 6 - protein, 98 aa, homo sapiens
QVQLVQSWAEVRKSGASVKVSCSFSGFTITSYGIHWVQQSPGQGLEWMGWINPGNGSPSYAKKFQGRFTMTRDMSTTTAYTDLSSLTSEDMAVYYYAR

SEQ ID NO: 7 - protein, 98 aa, homo sapiens
QMQLVQSGAEVKKTGSSVKVSCKASGYTFTYRYLHWVRQAPGQALEWMGWITPFNGNTNYAQKFQDRVTITRDRSMSTAYMELSSLRSEDTAMYYCAR

SEQ ID NO: 8 - protein, 98 aa, homo sapiens
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAR

SEQ ID NO: 9 - protein, 98 aa, homo sapiens
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAA

SEQ ID NO: 10 - protein, 98 aa, homo sapiens
QVQLGQSEAEVKKPGASVKVSCKASGYTFTCCSLHWLQQAPGQGLERMRWITLYNGNTNYAKKFQGRVTITRDMSLRTAYIELSSLRSEDSAVYYWAR

SEQ ID NO: 11 - protein, 98 aa, homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 12 - protein, 98 aa, homo sapiens
EVQLVQSGAEVKKPGATVKISCKVSGYTFTDYYMHWVQQAPGKGLEWMGLVDPEDGETIYAEKFQGRVTITADTSTDTAYMELSSLRSEDTAVYYCAT

SEQ ID NO: 13 - protein, 98 aa, homo sapiens; identical to SEQ ID NO: 11

SEQ ID NO: 14 - protein, 98 aa, homo sapiens
  misc_feature (46)..(46): Xaa can be any naturally occurring amino acid
  misc_feature (85)..(85): Xaa can be any naturally occurring amino acid
QVQLLQPGVQVKKPGSSVKVSCKASRYTFTKYFTRWVRQSPGQGHXWMGWINPYNDNTHYAQTFWGRVTITSDRSMSTAYMELSXLRSEDMVVYYCVR

SEQ ID NO: 15 - protein, 100 aa, homo sapiens
QITLKESGPTLVKPTQTLTLTCTFSGFSLSTSGVGVGWIRQPPGKALEWLALIYWNDDKRYSPSLKSRLTITKDTSKNQVVLTMTNMDPVDTATYYCAHR

SEQ ID NO: 16 - protein, 100 aa, homo sapiens
QVTLKESGPALVKPTQTLMLTCTFSGFSLSTSGMGVGWICQPSAKALEWLAHIYWNDNKYYSPSLKSRLIISKDTSKNEVVLTVINMDIVDTATHYCARR

SEQ ID NO: 17 - protein, 100 aa, homo sapiens
QVTLKESGPVLVKPTETLTLTCTVSGFSLSNARMGVSWIRQPPGKALEWLAHIFSNDEKSYSTSLKSRLTISKDTSKSQVVLTMTNMDPVDTATYYCARI

SEQ ID NO: 18 - protein, 100 aa, homo sapiens
QVTLRESGPALVKPTQTLTLTCTFSGFSLSTSGMCVSWIRQPPGKALEWLALIDWDDDKYYSTSLKTRLTISKDTSKNQVVLTMTNMDPVDTATYYCARI

SEQ ID NO: 19 - protein, 98 aa, homo sapiens
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAR

SEQ ID NO: 20 - protein, 99 aa, homo sapiens
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKD

SEQ ID NO: 21 - protein, 98 aa, homo sapiens
QVQLVESGGGLVKPGGSLRLSCAASGFTFSDYYMSWIRQAPGKGLEWVSYISSSGSTIYYADSVKGRFTISRDNAKNSLYLQMNSLRAEDTAVYYCAR

SEQ ID NO: 22 - protein, 96 aa, homo sapiens
VQLVESGGGLVQPGGSLRLSCAASGFTFSSYDMHWVRQATGKGLEWVSAIGTAGDTYYPGSVKGRFTISRENAKNSLYLQMNSLRAGDTAVYYCAR

SEQ ID NO: 23 - protein, 100 aa, homo sapiens
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTT

SEQ ID NO: 24 - protein, 98 aa, homo sapiens
EVQLVESGGGLVQPGGSLRLSCAASGFTFSNSDMNWARKAPGKGLEWVSGVSWNGSRTHYVDSVKRRFIISRDNSRNSLYLQKNRRRAEDMAVYYCVR

SEQ ID NO: 25 - protein, 98 aa, homo sapiens
TVQLVESGGGLVEPGGSLRLSCAASGFTFSNSDMNWVRQAPGKGLEWVSGVSWNGSRTHYADSVKGRFIISRDNSRNFLYQQMNSLRPEDMAVYYCVR

SEQ ID NO: 26 - protein, 98 aa, homo sapiens
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCAR

SEQ ID NO: 27 - protein, 98 aa, homo sapiens
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAR

SEQ ID NO: 28 - protein, 100 aa, homo sapiens
EVHLVESGGALVQPGGSLRLSCAASGFTFSYYYMSGVRQAPGKGLEWVGFIRNKANGGTTEYTTSVKGRFTISRDDSKSITYLQMKSLKTEDTAVYYCSR

SEQ ID NO: 29 - protein, 98 aa, homo sapiens
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAK

SEQ ID NO: 30 - protein, 98 aa, homo sapiens; identical to SEQ ID NO: 29

SEQ ID NO: 31 - protein, 98 aa, homo sapiens
EMQLVESGGGLQKPAWSPRLSCAASQFTFSSYYMNCVRQAPGNGLELVSQVNPNGGSTYLIDSGKDRFNTSRDNAKNTLHLQMNSLKTEDTALYYCTR

SEQ ID NO: 32 - protein, 97 aa, homo sapiens
EVELIEPTEDLRQPGKFLRLSCVASRFAFSSFWMSPVHQSAGKGLEWVIDIKDDGSQIHHADSVKGRFSISKDNAKNSLYLQMNSQRTEDMAVYGCT

SEQ ID NO: 33 - protein, 98 aa, homo sapiens
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCAR

SEQ ID NO: 34 - protein, 97 aa, homo sapiens
EVQLVESGEDPRQPGGSLRLSCADSGLTFSSYARNSVSQAPGKGLEWVVDIQCDGSQICYADSLKSKFTISKENAKNSLYLLMNSLRAAGTAVCYCM

SEQ ID NO: 35 - protein, 98 aa, homo sapiens; identical to SEQ ID NO: 33

SEQ ID NO: 36 - protein, 98 aa, homo sapiens
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAK

SEQ ID NO: 37 - protein, 97 aa, homo sapiens
EVELIESIEDLRQPGKFLRLSCVASRFAFSSFGMSRVHQSPGKGLEWVIDIKDDGSQIHHADSVKGRFSISKDNAKNSLYLQMNSQRAEDMDVYGCT

SEQ ID NO: 38 - protein, 97 aa, homo sapiens
EVQLVESGEDPRQPGGSLRLSCADSGLTFSSYGRSSVSQAPGKGLEWVVDIQCDGSQICYADSLKSKFTISKENAKNSLYLLMNSLRAEGTAVCYCM

SEQ ID NO: 39 - protein, 97 aa, homo sapiens
EVELIEPTEDLRQPGKFLRLSCVASRFAFSSFGMSPVHQSAGKGLEWVIDIKDDGSQIHHADSVKGRFSISKDNAKNSLYLQMNSQRTEDMAVYGCT

SEQ ID NO: 40 - protein, 97 aa, homo sapiens
EVQLVESGEDPRQPGGSLRLSCADSGLTFSSYGRNSVSQAPGKGLEWVVDIQCDGSQICYADSLKSKFTISKENAKNSLYLLMNSLRAAGTAVCYCM

SEQ ID NO: 41 - protein, 97 aa, homo sapiens
EVELIESIEDLRQPGKFLRLSCVASRFAFSSFGMSRVHQSPGKGLEWVIDIKDDGSQIHHADSVKGRFSISKDNAKNSLYLQMNTQRAEDVAVYGYT

SEQ ID NO: 42 - protein, 98 aa, homo sapiens
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAR

SEQ ID NO: 43 - protein, 97 aa, homo sapiens
EVQLVESGEDPRQPGGSLRLSCADSGLTFSSYGMSSVSQAPGKGLEWVVDIQCDGSQICYAQSVKSKFTISKENAKNSLYLQMNSLRAEGTAVCYCM

SEQ ID NO: 44 - protein, 98 aa, homo sapiens
EVQLVESGGGLVQPGGSLRLSCAASGFTFSNSDMNWVHQAPGKGLEWVSGVSWNGSRTHYADSVKGRFIISRDNSRNTLYLQTNSLRAEDTAVYYCVR

SEQ ID NO: 45 - protein, 97 aa, homo sapiens
EVQLVESGGGLVQPRGSLRLSCAASGFTVSSNEMSWIRQAPGKGLEWVSSISGGSTYYADSRKGRFTISRDNSKNTLYLQMNNLRAEGTAAYYCARY

SEQ ID NO: 46 - protein, 96 aa, homo sapiens
EVQLVESRGVLVQPGGSLRLSCAASGFTVSSNEMSWVRQAPGKGLEWVSSISGGSTYYADSRKGRFTISRDNSKNTLHLQMNSLRAEDTAVYYCKK

SEQ ID NO: 47 - protein, 99 aa, homo sapiens
EVQLVESGGVVVQPGGSLRLSCAASGFTFDDYTMHWVRQAPGKGLEWVSLISWDGGSTYYADSVKGRFTISRDNSKNSLYLQMNSLRTEDTALYYCAKD

SEQ ID NO: 48 - protein, 97 aa, homo sapiens
EDQLVESGGGLVQPGGSLRPSCAASGFAFSSYALHWVRRAPGKGLEWVSAIGTGGDTYYADSVMGRFTISRDNAKKSLYLHMNSLIAEDMAVYYCAR

SEQ ID NO: 49 - protein, 98 aa, homo sapiens
EVQLVESGGGLVQPGGSLRLSCAASGFTFSSYSMNWVRQAPGKGLEWVSYISSSSSTIYYADSVKGRFTISRDNAKNSLYLQMNSLRAEDTAVYYCAR

SEQ ID NO: 50 - protein, 100 aa, homo sapiens
EVQLVESGGGLVQPGRSLRLSCTASGFTFGDYAMSWFRQAPGKGLEWVGFIRSKAYGGTTEYTASVKGRFTISRDGSKSIAYLQMNSLKTEDTAVYYCTR

SEQ ID NO: 51 - protein, 98 aa, homo sapiens
EVQLVESGGGLVQPGGSLRLSCAASGFTFSSSWMHWVCQAPEKGLEWVADIKCDGSEKYYVDSVKGRLTISRDNAKNSLYLQVNSLRAEDMTVYYCVR

SEQ ID NO: 52 - protein, 97 aa, homo sapiens
EVQLVESGGGLIQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 53 - protein, 97 aa, homo sapiens
EVQLVESEENQRQLGGSLRLSCADSGLTFSSYYMSSDSQAPGKGLEWVVDIKQDRSQLCYAQSVKSRFTISKENAKNSLCLQMNSLRAEGTAVYYCM

SEQ ID NO: 54 - protein, 98 aa, homo sapiens
EVQLVESGEGLVQPGGSLRLSCAASGFTFSSSAMHWVRQAPRKGLEWVSVISTSGDTVLYTDSVKGRFTISRDNAQNSLSLQMNSLRAEGTVVYYCVK

SEQ ID NO: 55 - protein, 97 aa, homo sapiens
EVELIESIEGLRQLGKFLRLSCVASGFTFSSYAMSWVNETLGKGLEGVIDVKYDGSQIYHADSVKGRFTISKDNAKNSPYLQTNSLRAEDMTMHGCT

SEQ ID NO: 56 - protein, 98 aa, homo sapiens
EVQLVESGGGLVQPGGSLRLSCAASGFTFSSYAMHWVRQAPGKGLEYVSAISSNGGSTYYANSVKGRFTISRDNSKNTLYLQMGSLRAEDMAVYYCAR

SEQ ID NO: 57 - protein, 97 aa, homo sapiens
EVQLVESGGGLVQPGGSLRLSCAASGFTVSSNYMSWVRQAPGKGLEWVSVIYSGGSTYYADSVKGRFTISRDNSKNTLYLQMNSLRAEDTAVYYCAR

SEQ ID NO: 58 - protein, 97 aa, homo sapiens
EVQLVESGGGLVKPGGSLRLSCAASGFTFSDYYMNWVRQAPGKGLEWVSSISSSSTIYYADSVKGRFTISRDNAKNSLYLQMNSLRAEDTAVYYCAR

SEQ ID NO: 59 - protein, 100 aa, homo sapiens
EVQLVESGGGLVQPGGSLRLSCAASGFTFSDYYMSWVRQAPGKGLEWVGFIRNKANGGTTEYTTSVKGRFTISRDDSKSITYLQMNSLRAEDTAVYYCAR

SEQ ID NO: 60 - protein, 100 aa, homo sapiens
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAR

SEQ ID NO: 61 - protein, 100 aa, homo sapiens
EVQLVESGGGLVQPGGSLKLSCAASGFTFSGSAMHWVRQASGKGLEWVGRIRSKANSYATAYAASVKGRFTISRDDSKNTAYLQMNSLKTEDTAVYYCTR

SEQ ID NO: 62 - protein, 98 aa, homo sapiens
EVQLVESGGGLVQPGGSLRLSCAASGFTFSSYWMHWVRQAPGKGLVWVSRINSDGSSTSYADSVKGRFTISRDNAKNTLYLQMNSLRAEDTAVYYCAR

SEQ ID NO: 63 - protein, 98 aa, homo sapiens
QVQLVESGGGVVQPGGSLRLSCAASGFTFSSYGMHWVRQAPGKGLEWVSVIYSGGSSTYYADSVKGRFTISRDNSKNTLYLQMNSLRAEDTAVYYCAK

SEQ ID NO: 64 - protein, 98 aa, homo sapiens
QVQLQESGPGLVKPPGTLSLTCAVSGGSISSSNWWSWVRQPPGKGLEWIGEIYHSGSTNYNPSLKSRVTISVDKSKNQFSLKLSSVTAADTAVYCCAR

SEQ ID NO: 65 - protein, 98 aa, homo sapiens
QVQLQESGPGLVKPSDTLSLTCAVSGYSISSSNWWGWIRQPPGKGLEWIGYIYYSGSTYYNPSLKSRVTMSVDTSKNQFSLKLSSVTAVDTAVYYCAR

SEQ ID NO: 66 - protein, 99 aa, homo sapiens
QLQLQESGSGLVKPSQTLSLTCAVSGGSISSGGYSWSWIRQPPGKGLEWIGYIYHSGSTYYNPSLKSRVTISVDRSKNQFSLKLSSVTAADTAVYYCAR

SEQ ID NO: 67 - protein, 99 aa, homo sapiens
QVQLQESGPGLVKPSQTLSLTCTVSGGSISSGDYYWSWIRQPPGKGLEWIGYIYYSGSTYYNPSLKSRVTISVDTSKNQFSLKLSSVTAADTAVYYCAR

SEQ ID NO: 68 - protein, 99 aa, homo sapiens
QVQLQESGPGLVKPSQTLSLTCTVSGGSISSGGYYWSWIRQHPGKGLEWIGYIYYSGSTYYNPSLKSLVTISVDTSKNQFSLKLSSVTAADTAVYYCAR

SEQ ID NO: 69 - protein, 97 aa, homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCAR

SEQ ID NO: 70 - protein, 98 aa, homo sapiens
QVQLQESGPGLVKPSETLSLTCAVSGYSISSGYYWGWIRQPPGKGLEWIGSIYHSGSTYYNPSLKSRVTISVDTSKNQFSLKLSSVTAADTAVYYCAR

SEQ ID NO: 71 - protein, 99 aa, homo sapiens
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCAR

SEQ ID NO: 72 - protein, 97 aa, homo sapiens
QVQLQESGPGLVKPSETLSLICAVSGDSISSGNWGWVRQPPGKGLEWIGEIHHSGSTYYNPSLKSRITMSVDTSKNQFYLKLSSVTAADTAVYYCAR

SEQ ID NO: 73 - protein, 97 aa, homo sapiens
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCAR

SEQ ID NO: 74 - protein, 99 aa, homo sapiens
QVQLQESGPGLVKPSETLSLTCTVSGGSVSSGSYYWSWIRQPPGKGLEWIGYIYYSGSTNYNPSLKSRVTISVDTSKNQFSLKLSSVTAADTAVYYCAR

SEQ ID NO: 75 - protein, 98 aa, homo sapiens
EVQLVQSGAEVKKPGESLRISCKGSGYSFTSYWISWVRQMPGKGLEWMGRIDPSDSYTNYSPSFQGHVTISADKSISTAYLQWSSLKASDTAMYYCAR

SEQ ID NO: 76 - protein, 98 aa, homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 77 - protein, 98 aa, homo sapiens
EVQLLQSAAEVKRPGESLRISCKTSGYSFTSYWIHWVRQMPGKELEWMGSIYPGNSDTRYSPSFQGHVTISADSSSSTAYLQWSSLKASDAAMYYCVR

SEQ ID NO: 78 - protein, 101 aa, homo sapiens
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAR

SEQ ID NO: 79 - protein, 98 aa, homo sapiens
QVQLVQSGSELKKPGASVKVSCKASGYTFTSYAMNWVRQAPGQGLEWMGWINTNTGNPTYAQGFTGRFVFSLDTSVSTAYLQICSLKAEDTAVYYCAR

SEQ ID NO: 80 - protein, 98 aa, homo sapiens
QLQLVQSGPEVKKPGASVKVSYKSSGYTFTIYGMNWVRQTPGQGFEWMGWIITYTGNPTYTHGFTGWFVFSMDTSVSTACLQISSLKAEDTAEYYCAK

SEQ ID NO: 81 - protein, 98 aa, homo sapiens
QVQLVQSGHEVKQPGASVKVSCKASGYSFTTYGMNWVPQAPGQGLEWMGWFNTYTGNPTYAQGFTGRFVFSMDTSASTAYLQISSLKAEDMAMYYCAR

SEQ ID NO: 82 - protein, 98 aa, homo sapiens
EAQLTESGGDLVHPEGPLRLSCAASWFTFSIYEIHWVCQASGKGLEWVAVIWRSESHQYNADYVRGRLTTSRDNTKYMLYMQMNSLRTQNMAAFNCAG

(19) United States
(12) Patent Application Publication
(10) Pub. No.: US 2021/0130440 A1
(43) Pub. Date: May 6, 2021

(54) METHODS FOR PRODUCING BIOTHERAPEUTICS WITH INCREASED STABILITY BY SEQUENCE OPTIMIZATION

(71) Applicant: Janssen Biotech, Inc., Horsham, PA (US)

(72) Inventors: Sathyadevi Venkataramani, Blue Bell, PA (US); Rajkumar Ganesan, Blue Bell, PA (US); Sanjaya Singh, Blue Bell, PA (US)

(21) Appl. No.: 17/061,590

(22) Filed: Oct. 2, 2020

Related U.S. Application Data
(60) Provisional application No. 62/909,841, filed on Oct. 3, 2019.

(51) Int. Cl. C07K 16/00; G16B 30/10; G16B 20/20; G16B 15/20

(52) U.S. Cl. CPC C07K 16/00; G16B 30/10; G16B 20/20; C07K 2317/94; C07K 2317/92; C07K 2317/56; G16B 15/20

(57) ABSTRACT

The invention relates to methods of optimizing an antibody with enhanced stability, the method comprising mutating somatic hypermutation with germline amino acid residues and therefore providing enhanced thermal stability, improved biophysical properties and shelf-life while preserving the affinity for the antigen.

### CROSS-REFERENCE TO RELATED APPLICATIONS

This application claims priority to U.S. Application No. 62/909,841, filed Oct. 3, 2019, the disclosure of which is herein incorporated by reference in its entirety.

### SEQUENCE LISTING

The instant application contains a Sequence Listing which has been submitted electronically in ASCII format and is hereby incorporated by reference in its entirety. The ASCII copy, created on Dec. 3, 2020, is named JBI6147USNP1_SL_ST25.txt and is 75,288 bytes in size.

### TECHNICAL FIELD

This invention relates to a method of optimizing the sequence of a monoclonal antibody to enhance its biophysical properties, including thermodynamic stability for optimized manufacturing, in vivo behavior and longer shelf-life.

### BACKGROUND OF THE INVENTION

Antibodies are generated as a protective response by the immune system generally triggered after exposure to an antigen. Although, the antibodies made in the primary response following exposure of antigen are of lower affinity, the affinities are known to be improved by a process called somatic immunoglobulin (Ig) hypermutation (Neuberger, M. S. & Milstein, C. Somatic hypermutation. Current opinion in immunology 7, 248-254 (1995). The process involves several rounds of recombination of immunoglobulin gene segments; variable (V), diversity (D), and joining (J) along with the accumulation of set of mutations in the complementarity determining regions (CDR's) of the antibody that result in antibodies with very strong affinities and high selectivity to the antigen (Sun, S. B. et al. Mutational analysis of 48G7 reveals that somatic hypermutation affects both antibody stability and binding affinity, Journal of the American Chemical Society 135, 9980-9983 (2013); Retracted by authors on the basis of duplicated data). While most of these accumulated mutations are present in the CDR's and are crucial for high-affinity interaction with the antigen, a few others are spread throughout the variable regions and do not participate in direct antigen binding and thus do not contribute to antibody affinity (Wang, F. et al. Somatic hypermutation maintains antibody thermodynamic stability during affinity maturation. Proceedings of the National Academy of Sciences of the United States of America 110, 4261-4266 (2013); Retracted by authors on the basis of duplicated data). CDRs (also often called hypervariable regions) are loops in the variable regions of both heavy and light chains that form the sites for antibody antigen recognition. Their conformations are defined by the length and composition of the loops. Framework (FR) regions are anti-parallel β-strands arranged into two β-sheets that are responsible for maintaining the structural integrity of the variable domains and serve as a structural scaffold for CDRs. FRs are important for structural diversity, VL/VH orientation, and may also be involved directly in antigen binding (Sela-Culang, I., et al. The Structural Basis of Antibody-Antigen Recognition. Frontiers in Immunology 4 (2013). Affinity mutations occurring in the CDR's during affinity maturation can have deleterious effects on antibody stability. It has recently been shown that neutral somatic hypermutations, located throughout the variable regions, often compensate for the adverse effects on antibody stability caused by affinity mutations (Sun, S. B. et al. Mutational analysis of 48G7 reveals that somatic hypermutation affects both antibody stability and binding affinity. Journal of the American Chemical Society 135, 9980-9983 (2013)). This strong trade-off between affinity and stability of antibody scaffolds has also been shown in directed evolution studies where mutations acquired during the affinity maturation process either within the CDRs or in the framework regions can be functional and simultaneously destabilizing (Houlihan, G., Gatti-Lafranconi, P., Lowe, D. & Hollfelder, F. Directed evolution of anti-HER2 DARPins by SNAP display reveals stability/function trade-offs in the selection process. Protein engineering, design & selection: PEDS 28, 269-279 (2015); Julian, M. C. et al. Co-evolution of affinity and stability of grafted amyloid-motif domain antibodies. Protein engineering, design & selection: PEDS 28, 339-350 (2015)).

Antibodies and related products are the fastest growing class of therapeutic agents. Therapeutic antibodies must exhibit favorable pharmaceutical properties, including high thermostability and low aggregation propensity, in order to facilitate manufacturing and storage, as well as to promote long serum half-life. Functionally active molecules can become drugs only if they possess favorable biophysical properties which include conformational and colloidal stability. (Jain, T. et al. Biophysical properties of the clinical-stage antibody landscape. Proceedings of the National Academy of Sciences of the United States of America 114, 944-949 (2017)). Conformational stability of antibodies is dictated, for example, by higher thermal stability and lower propensity to aggregate. Thermal stability plays a key role in drug discovery starting from antibody expression, purification, formulation and shelf-life (Goswami, S., Wang, W., Arakawa, T. & Ohtake, S. Developments and Challenges for mAb-Based Therapeutics. Antibodies 2, 452-500 (2013)). High throughput automated screening assays are critical to determine the conformational stability and to rank order hundreds of hits early in development.

Enhanced thermal stability is key for optimal pharmacokinetic and pharmacodynamic properties, and longer shelf-life and storage (Thiagarajan, G., Semple, A., James, J. K., Cheung, J. K. & Shameem, M. A comparison of biophysical characterization techniques in predicting monoclonal antibody stability. MAbs 8, 1088-1097 (2016)). It has been consistently observed that in vitro affinity matured antibodies are less thermostable than their parental antibody. Further optimization through a combination of CDR-grafting onto the stable framework, mammalian cell display and in vitro somatic hypermutation (SHM) is often required to improve stability of the antibodies (McConnell, A. D. et al. A general approach to antibody thermostabilization. MAbs 6, 1274-1282 (2014)).

The prevention of adverse side effects is crucial for the safety of the patient and the success of a biotherapeutic candidate. It is of utmost importance to assess expected immunogenicity in the earliest developmental stages of an antibody drug candidate and to eliminate potential adverse effects. Different methods were developed to assess and reduce the expected immunogenicity, as rigorous pre-clinical risk assessment is required by the FDA.

Antibody engineering technologies are pivotal for discovery and development of biotherapeutics. Antibodies discovered from non-human species are humanized to overcome and reduce the risk of immunogenicity. Humanization of antibodies derived from non-human species are being applied successfully to optimize clinical development of mAbs. Humanized antibodies represent ˜43% (i.e. 38 mAbs) of the 89 FDA currently approved antibodies. Fully human antibodies are increasingly common and are a growing proportion of mAbs in the clinic. They are being sourced from transgenic animals genetically engineered with humanized humoral immune system, such as XenoMouse®, Ablexis® and OmniRat®. To date, 21 fully human mAbs obtained from transgenic animals have been approved, which corresponds to 24% of all the marketed mAbs. Some of the antibody sequences obtained from transgenic animals contain somatic hypermutations in the framework and CDR regions. Somatic hypermutations may result in unusual or low frequency residues in human framework regions and impact the stability and immunogenicity of biotherapeutics.

Generating stable antibodies with long shelf-life and low immunogenicity remains challenging and is often a long and painful process.

### BRIEF SUMMARY OF THE INVENTION

In certain embodiments, the invention provides a method of designing optimized antibodies, the method comprising:
a) Identifying an antibody for optimizing;
b) Identifying one or more unusual or low frequency residues in said antibody VH and/or VL;
c) Aligning said antibody VH and/or VL sequences with the closest human and non-human germline sequences;
d) Identifying one or more somatic hypermutation sites in said antibody VH, VL, or both;
e) Identifying one or more germline residues typically observed at the site of said somatic hypermutation sites;
f) Designing and engineering variants or a library of variants containing said germline residues at the site of said somatic hypermutation sites;
g) Assessing properties of said variants or library of variants; and
h) Selecting one or more optimized variants,
wherein said one or more optimized variants has improved biophysical properties, decreased risk of immunogenicity, or both.

In other certain embodiments, the invention provides a method of designing optimized antibodies, the method comprising:
a) Identifying an antibody for optimizing;
b) Identifying one or more unusual or low frequency residues in said antibody VH and/or VL;
c) Aligning said antibody VH and/or VL sequences with the closest human and non-human germline sequences;
d) Identifying one or more somatic hypermutation sites in said antibody VH, VL, or both;
e) Identifying one or more germline residues typically observed at the site of said somatic hypermutation sites;
f) Designing and engineering variants or a library of variants containing said germline residues at the site of said somatic hypermutation sites; Cloning and producing said variants or library of variants;
h) Assessing biophysical properties of said variants or library of variants;
i) Assessing immunogenicity risks of said variants or library of variants; and
j) Selecting one or more optimized variants, wherein said one or more optimal optimized variants has improved biophysical properties, decreased risk of immunogenicity, or both.

In certain embodiments, the identification of unusual or low frequency residues is done, for example, by a computer-based software, such as but not limited to abYsis.

In certain embodiments, the biophysical assessment is done by, for example, analytical ultracentrifugation, thermal stability, free energy of unfolding, or analytical size exclusion.

In certain embodiments, the immunogenicity risk assessment is done, for example, in-silico, such as by Epivax® score.

In certain embodiments, the amino acid replacements can occur, for example, in any one or more of the four FRs. In this respect, the amino acid replacements can occur in FR1, FR2, FR3, and/or FR4 of the heavy or light chain. In other certain embodiment, the one or more amino acid residues can be replaced with a germline residue, so long as the amino acid replacements improve the biophysical properties of the optimized antibody.

In some other embodiments, the amino acid replacements can occur, for example, in any one or more of the CDRs. In this respect, the amino acid replacements can occur in CDR1, CDR2 and/or CDR3 of the heavy or light chain. In other certain embodiments, the one or more amino acid residues can be replaced with a germline residue, so long as the amino acid replacements improve the biophysical properties of the optimized antibody.

In other embodiments, the invention is not limited to an isolated antigen-binding agent comprising an antibody heavy chain polypeptide or light chain polypeptide. Indeed, any amino acid residue of the framework resulting from somatic hypermutation, can be replaced, in any combination, with a germline amino acid residue, as long as the stability of the antigen-binding agent is enhanced or improved as a result of the amino acid replacement without concomitant loss of biological activity.

### BRIEF DESCRIPTION OF THE DRAWINGS

FIG. 1A shows the sequence alignment of TMEB675 with human germline sequences for VH and the identification of unusual or low frequency residues. Three somatic hypermutations (SHM) in VH (R14P, P20L, H81Q) were observed within the framework region.

FIG. 1B shows the sequence alignment of TMEB675 with human germline sequences for Vk and the identification of unusual or low frequency residues. One somatic hypermutation (SHM) (A1D) was observed in the framework region and one in the CDR3 (A91P).

FIG. 2A shows the assessment of the relative frequency of the SHM Arginine (R) at position 14 of TMEB675 VH using the abYsis portal.

FIG. 2B shows the assessment of the relative frequency of the SHM Proline (P) at position 20 of TMEB675 VH using the abYsis portal.

FIG. 2C shows the assessment of the relative frequency of the SHM Histidine (H) at position 81 of TMEB675 VH using the abYsis portal.

FIG. 2D shows the assessment of the relative frequency of the SHM Alanine (A) at position 1 of TMEB675 VL using the abYsis portal.

FIG. 2E shows the assessment of the relative frequency of the SHM Alanine (A) at position 91 of TMEB675 VL using the abYsis portal.

FIG. 3 shows a molecular homology model of TMEB675. SHM residues found in the framework regions are labeled and highlighted in the stick representation.

FIG. 4 shows normalized g(s*) sedimentation velocity runs by analytical ultra-centrifugation for both TMEB675 and TMEB762. Global fitting analysis was done by SEDANAL v697 and the data was fitted globally to two species, non-interacting model.

FIG. 5 shows Intrinsic Properties characterization of TMEB675 and TMEB762 using Differential Scanning Fluorimetry (DSF). First derivative intensity of the 350/330 nm ratio is plotted against the temperature (° C.).

FIG. 6 shows Intrinsic Properties Characterization of TMEB675 and TMEB762 using Differential Scanning calorimetry (DSC). Heat capacity Cp (cal/mol/° C.) is plotted against the temperature (° C.).

FIG. 7 shows Intrinsic Properties Characterization of TMEB675 using Isothermal Chemical Denaturation in GdnCl as monitored by change in the fluorescence intensity ratio 350/330 nM.

FIG. 8 shows Intrinsic Properties Characterization of TMEB762 using Isothermal Chemical Denaturation in GdnCl as monitored by change in the fluorescence intensity ratio 350/330 nM.

FIG. 9 shows storage (4° C.) and accelerated (40° C.) stability of TMEB762 and TMEB675 over a month. Change in aggregate level between time zero and 1 month is plotted against days.

FIG. 10 shows non-specific binding data for TMEB762 and TMEB675 as determined by surface plasmon resonance method by plotting relative binding response units to different surfaces.

FIG. 11 shows the optimization algorithm workflow.

FIG. 12 shows sequence alignment of heavy chain (VH) of PSMW56, human germline IGHV4-39*01 and PSMW57. The rare somatic hypermutation at position 68 (Threonine to Isoleucine) is highlighted in bold. PSMW57 is an engineered variant of PSMW56. Ile68 was germlined to Threonine.

FIG. 13 shows the unusual or low frequency framework residue (Ile) at position 68. This residue was re-engineered into a Thr residue. The choice of Thr was based on the germline residue (IGHV4-39*01).

FIG. 14 shows Intrinsic Properties Characterization of PSMW56 and PSMW57 using Differential Scanning Fluorimetry (DSF). The engineered variant PSMW57 shows significantly improved Tm and Tagg, compared to the parental variant PSMW56.

FIG. 15A shows sequence alignment of the DL3B355 heavy chain, with the human germlines (IGHV3-13*05) and the engineered variants: DL3B355-1, DL3B355-2 and DL3B355-3. HCDR1, HCDR2 and HCDR3 sequences are underlined. The rare somatic hypermutation at position 85 (Histidine) is highlighted in bold.

FIG. 15B shows sequence alignment of the DL3B355 light chain, with the human germlines (IGKV1-5*03) and the engineered variants: DL3B355-1, DL3B355-2 and DL3B355-3. LCDR1, LCDR2 and LCDR3 sequences are underlined. The rare somatic hypermutation at position 84 (Glu) is highlighted in bold.

FIG. 16A shows the unusual or low frequency framework residue at heavy chain position 85 of DL3B355. This residue was re-engineered to match with the corresponding germline residue (Asparagine).

FIG. 16B shows the unusual or low frequency framework residue at light chain at position 84 of DL3B355. This residue was re-engineered to match with the corresponding germline residue (Glycine).

FIG. 17 shows Intrinsic Properties Characterization of DL3B355 and DLL3 variants using Differential Scanning Fluorimetry (DSF). All three engineered DL3B355 variants showed improved thermal stability (Tm and Tagg) compared the parental clone.

### DETAILED DESCRIPTION OF THE INVENTION

The foregoing summary, as well as the following detailed description of embodiments of the present application, will be better understood when read in conjunction with the appended drawings. It should be understood, however, that the application is not limited to the precise embodiments shown in the drawings.

Discussion of documents, acts, materials, devices, articles or the like which has been included in the present specification is for the purpose of providing context for the invention. Such discussion is not an admission that any or all of these matters form part of the prior art or limit any invention disclosed or claimed.

The numbering of amino acid residues of the antibody constant region throughout the specification is according to the EU index as described in Kabat et al., Sequences of Proteins of Immunological Interest, 5th Ed. Public Health Service, National Institutes of Health, Bethesda, Md. (1991), unless otherwise explicitly stated.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, exemplary materials and methods are described herein. In describing and claiming the present invention, the following terminology will be used.

As used in this specification and the appended claims, the singular forms “a,” “an,” and “the” include plural referents unless the content clearly dictates otherwise. Thus, for example, reference to “a cell” includes a combination of two or more cells, and the like.

The transitional terms “comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristic(s)” of the claimed invention. Embodiments described in terms of the phrase “comprising” (or its equivalents) also provide as embodiments those independently described in terms of “consisting of” and “consisting essentially of.”

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

It should also be understood that the terms “about,” “approximately,” “generally,” “substantially,” and like terms, used herein when referring to a dimension or characteristic of a component of the invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

Unless otherwise stated, any numerical values, such as a concentration or a concentration range described herein, are to be understood as being modified in all instances by the term “about.” “About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 10%, whichever is greater. Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

“Antigen” refers to any molecule (e.g., protein, peptide, polysaccharide, glycoprotein, glycolipid, nucleic acid, portions thereof, or combinations thereof) that is capable of mediating an immune response. Exemplary immune responses include antibody production and activation of immune cells, such as T cells, B cells or NK cells.

“Antigen binding fragment” or “antigen binding domain” refers to a portion of a protein that binds the antigen. Antigen binding fragments may be synthetic, enzymatically obtainable or genetically engineered polypeptides and include portions of an immunoglobulin that bind an antigen, such as a VH, a VL, the VH and the VL, Fab, Fab′, F(ab′)₂, Fd and Fv fragments, domain antibodies (dAb) consisting of one VH domain or one VL domain, camelized VH domains, VHH domains, minimal recognition units consisting of the amino acid residues that mimic the CDRs of an antibody, such as FR3-CDR3-FR4 portions, the HCDR1, the HCDR2 and/or the HCDR3 and the LCDR1, the LCDR2 and/or the LCDR3, alternative scaffolds that bind an antigen, and multispecific proteins comprising the antigen binding fragments. Antigen binding fragments (such as the VH and the VL) may be linked together via a synthetic linker to form various types of single antibody designs in which the VH/VL domains may pair intramolecularly, or intermolecularly in those cases when the VH and the VL domains are expressed by separate single chains, to form a monovalent antigen binding domain, such as single chain Fv (scFv) or diabody. Antigen binding fragments may also be conjugated to other antibodies, proteins, antigen binding fragments or alternative scaffolds which may be monospecific or multispecific to engineer bispecific and multispecific proteins.

“Antibody” is meant in a broad sense and includes immunoglobulin molecules including monoclonal antibodies including murine, human, humanized and chimeric monoclonal antibodies, antigen binding fragments, multispecific antibodies, such as bispecific, trispecific, tetraspecific etc., dimeric, tetrameric or multimeric antibodies, single chain antibodies, domain antibodies and any other modified configuration of the immunoglobulin molecule that comprises an antigen binding site of the required specificity. “Full length antibodies” are comprised of two heavy chains (HC) and two light chains (LC) inter-connected by disulfide bonds as well as multimers thereof (e.g. IgM). Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDR), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4. Immunoglobulins may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (λ), based on the amino acid sequences of their constant domains.

“Biological activity” refers to, for example, binding affinity, neutralization or inhibition of antigen.

“Complementarity determining regions” (CDR) are antibody regions that bind an antigen. There are three CDRs in the VH (HCDR1, HCDR2, HCDR3) and three CDRs in the VL (LCDR1, LCDR2, LCDR3). CDRs may be defined using various delineations such as Kabat (Wu et al., (1970) J Exp Med 132(2): 211-250), (Kabat et al. (1991, J Immunol 147(5): 1709-19), Chothia (Chothia et al, (1987) J. Mol. Biol. 196(4):901-17, IMGT (Lefranc et al., (2003) Dev Comp Immunol 27(1): 55-77) and AbM (Martin and Thornton (1996) J Mol Biol 263(5): 800-815). The correspondence between the various delineations and variable region numbering is described (see e.g. Lefranc et al. (2003) Dev Comp Immunol 27(1): 55-77; Honegger and Pluckthun, J Mol Biol (2001) 309(3):657-670; International ImMunoGeneTics (IMGT) database; Web resources, http://www_imgt_org). Available programs such as abYsis by UCL Business PLC may be used to delineate CDRs. The term “CDR”, “HCDR1”, “HCDR2”, “HCDR3”, “LCDR1”, “LCDR2” and “LCDR3” as used herein includes CDRs defined by any of the methods described supra, Kabat, Chothia, IMGT or AbM, unless otherwise explicitly stated in the specification.

“Framework region” or “FR” are antibody regions that act as scaffold for the CDRs. The framework region is responsible for supporting the binding of the antigen to the antibody. Framework residues comprise residues that come in contact with the antigen are a part of the antibody's binding site and are located either close in sequence to the CDRs or in close proximity to the CDR when in the folded three-dimensional structure. Framework residues also comprise residues that do not come in contact with the antigen but affect the binding indirectly by aiding in structural support for the CDR. FRs may be defined using various delineations such as Kabat, Chothia, IMGT and AbM (Martin and Thornton (1996) J Mol Biol 263: 800-815). Available programs such as abYsis by UCL Business PLC may be used to delineate FRs. The term “FR1”, “FR2”, “FR3”, “FR4”, includes FRs defined by any of the methods described above. The term “HCFR” represents the heavy chain framework regions FR1, FR2, FR3 or FR4. The term “LCFR” represents the light chain framework regions FR1, FR2, FR3 or FR4.

“Immunoglobulins” may be assigned to five major classes, IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4. Antibody light chains of any vertebrate species may be assigned to one of two clearly distinct types, namely kappa (κ) and lambda (k), based on the amino acid sequences of their constant domains.

“Human antibody” refers to an antibody that is optimized to have minimal immune response when administered to a human subject. Variable regions of human antibody are derived from human immunoglobulin sequences. If human antibody contains a constant region or a portion of the constant region, the constant region is also derived from human immunoglobulin sequences. Human antibody comprises heavy and light chain variable regions that are “derived from” sequences of human origin if the variable regions of the human antibody are obtained from a system that uses human germline immunoglobulin or rearranged immunoglobulin genes. Such exemplary systems are human immunoglobulin gene libraries displayed on phage, and transgenic non-human animals such as mice or rats carrying human immunoglobulin loci. “Human antibody” typically contains amino acid differences when compared to the immunoglobulins expressed in humans due to differences between the systems used to obtain the human antibody and human immunoglobulin loci, introduction of somatic mutations or intentional introduction of substitutions into the frameworks or CDRs, or both. Typically, “human antibody” is at least about 80%, 81%, 82%, 83%, 84%, 85%, 86%, 87%, 88%, 89%, 90%, 91%, 92%, 93%, 94%, 95%, 96%, 97%, 98% or 99% identical in amino acid sequence to an amino acid sequence encoded by human germline immunoglobulin or rearranged immunoglobulin genes. In some cases, “human antibody” may contain consensus framework sequences derived from human framework sequence analyses, for example as described in Knappik et al., (2000) J Mol Biol 296:57-86, or a synthetic HCDR3 incorporated into human immunoglobulin gene libraries displayed on phage, for example as described in Shi et al., (2010) J Mol Biol 397:385-96, and in Int. Patent Publ. No. WO2009/085462. Antibodies in which at least one CDR is derived from a non-human species are not included in the definition of “human antibody”.

“Humanized antibody” refers to an antibody in which at least one CDR is derived from non-human species and at least one framework is derived from human immunoglobulin sequences. Humanized antibody may include substitutions in the frameworks so that the frameworks may not be exact copies of expressed human immunoglobulin or human immunoglobulin germline gene sequences.

“Isolated” refers to a homogenous population of molecules (such as scFv of the disclosure or heterologous proteins comprising the scFv of the disclosure) which have been substantially separated and/or purified away from other components of the system the molecules are produced in, such as a recombinant cell, as well as a protein that has been subjected to at least one purification or isolation step. “Isolated” refers to a molecule that is substantially free of other cellular material and/or chemicals and encompasses molecules that are isolated to a higher purity, such as to 80%, 81%, 82%, 83%, 84%, 85%, 86%, 87%, 88%, 89%, 90%, 91%, 92%, 93%, 94%, 95%, 96%, 97%, 98%, 99% or 100% purity.

“Variant,” “mutant” or “altered” refers to a polypeptide or a polynucleotide that differs from a reference polypeptide or a reference polynucleotide by one or more modifications, for example one or more substitutions, insertions or deletions. More concretely, the invention relates to a variant polypeptide, wherein the variant has an amino acid sequence which, when aligned with the germline immunoglobulin sequence comprises at least one substitution of an amino acid residue corresponding to any amino acids in FR1, FR2, FR3, FR4, CDR1, CDR2 or CDR3 and wherein the substitution site is a SMH site identified in the lead antibody. A variant polypeptide may have an improved property as compared to a reference polypeptide, in particular with respect to a property relevant to stability. Improved stability may be demonstrated by a variant that shows improved thermal stability, increased energy of unfolding, lower aggregation, improved storage stability or improved non-specific binding properties. The improved property will typically be a property with relevance to the use of the variant antibody in manufacturing. In some embodiment of the invention, the modification site is identified through sequence alignment with germline antibodies. In a particular embodiment, the sequence alignment was done with the software abYsis (Swindells, M. B. et al. abYsis: Integrated Antibody Sequence and Structure-Management, Analysis, and Prediction. J Mol Biol 429, 356-364 (2017)). In some embodiment, the modification substitutions, insertions or deletions is done by antibody engineering techniques.

“Tm” or “mid-point temperature” “is the temperature midpoint of a thermal unfolding curve. It refers to the temperature where 50% of the amino acid sequence is in its native conformation and the other 50% is denatured. A thermal unfolding curve is typically plotted as a function of temperature. Tm is used to measure protein stability. In general, a higher Tm is an indication of a more stable protein. The Tm can be readily determined using methods well known to those skilled in the art such as Circular Dichroism Spectroscopy, Differential Scanning calorimetry, Differential Scanning Fluorimetry (both intrinsic and extrinsic dye based), UV spectroscopy, FT-IR and Isothermal calorimetry (ITC).

“Tagg” refers to the temperature at which the protein starts to aggregate either through dimerization or oligomerization. The aggregation temperature detects the onset of aggregation, the temperature at which a protein will show a tendency to aggregate. Tagg can be determined by differential scanning calorimetry (DSC), Differential Scanning Fluorimetry (DSF) or by circular dichroism (CD). These techniques can detect small changes in the conformation of the protein and therefore detect the starting point of aggregation. Tagg values can be lower or higher than Tm. In cases where Tagg is lower than Tm, the protein either dimerizes and/or oligomerizes first and then starts unfolding later at higher temperatures than the Tagg. In cases where Tagg is higher than Tm, the protein starts to unfold first and then aggregates at a higher temperature than the Tm. Both events are commonly observed and depend on amino acid composition and protein conformation.

Chemical denaturation is a perfect complementary method to thermal denaturation for measuring intrinsic stability of proteins even at lower temperatures (4° C. to 40° C., storage and physiological temperatures) eliminating the need of extrapolating stability values from higher temperatures. Temperature extrapolations are highly prone to error since the temperature dependent stability of a protein is a function of three important parameters such as ΔH, the enthalpy of unfolding, ΔS, the entropy of unfolding and ΔCp and the heat capacity change of unfolding.

“ΔGᵤ” which refers to the “Gibbs free energy of unfolding” plays a critical role in determining the intrinsic stability of proteins at lower temperatures. ΔGᵤ is measured by chemical denaturation. Determination of ΔGᵤ is used for stability optimization and aggregation minimization. Proteins with higher ΔGᵤ, are more stable in their native conformation. The presence of even a small amount of denatured protein at lower temperatures can trigger aggregation, chemical degradation and hence loss of binding and function. It is therefore critical to determine the free energy of unfolding for therapeutic candidates to understand the stability of their native conformation. Chemical denaturation can be measured in the presence of denaturants such as guanidium chloride and/or urea by techniques such as ultraviolet, fluorescence and Circular Dichroism Spectroscopic methods. Three parameters (ΔGᵤ, C₅₀ and m) are determined by nonlinear least-squares fitting of the data collected from chemical denaturation where m is the rate of change in ΔGᵤ as a function of denaturant concentration and C₅₀ is the denaturant concentration at which 50% of protein molecules are in the native folded state and 50% in unfolded denatured state. Increase in both ΔGᵤ and C₅₀ denotes the increase in intrinsic stability of proteins. In cases two unfolding transitions are observed, ΔGu1 and ΔGu2 will refer to the first unfolding transition and the second unfolding transition, respectively

“Improved stability” refers to an antibody variant with increase tolerance to high or low temperature, immunoglobulin aggregation, and other stresses tested during antibody manufacturing. The antibody of the disclosure having improved stability is an antibody with an increase in monomer content, an elevated melting point (Tm), an elevated Tagg, an elevated free-energy of unfolding (ΔGu1, ΔGu1, C₅₀), or a reduced level of aggregation when compared to the same antibody differing only in one or more somatic hypermutation sites. The elevation in monomer content may be by 2% or more. The elevated Tm may be an elevation of 1° C. or more, such as 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. The elevated Tagg may be an elevation of 1° C. or more, such as 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C. The elevated ΔGu1 (first unfolding transition) or ΔGu2 (second unfolding transition) may be an increase of 4 kJ/mol or more. The increase in C₅₀ may be by 0.1 M or more. The decrease in aggregation may be 1% or more.

“Surface exposed” refers to an amino acid residue that is at least partially exposed to a surface of a protein and accessible to solvent, such as accessible to deuteriation. Algorithms are well-known in the art for predicting surface accessibility of residues based on primary sequence or a protein. Alternatively, surface exposed residues may be identified from a crystal structure of the protein.

“Somatic hypermutation” or “SHM” refers to the mutation of a polynucleotide sequence which can be initiated by, or associated with, the action of a cellular mechanism by which the immune system adapts to the new foreign elements, as seen during class switching. A major component of the process of affinity maturation, SHM diversifies B cell receptors used to recognize foreign elements such as antigens and allows the immune system to adapt its response to new threats during the lifetime of an organism. Somatic hypermutation affects the variable region of immunoglobulin genes. The present invention provides a method of increasing the stability of an antibody, the method comprises the step of identifying somatic hypermutation in the framework or CDR regions of an antibody through sequence alignment with a germline antibody. The method further comprises the step of assessing the frequency of the amino acid residue present at the SHM site and mutating it to a corresponding germline amino acid.

“Germline antibodies” are antibody sequences encoded by non-lymphoid cells that have not undergone the maturation process that leads to genetic rearrangement and mutation for expression of a particular antibody. One of the advantages provided by various embodiments of the present invention stems from the recognition that germline antibody genes are more likely than mature antibody genes to conserve essential amino acid sequence structures characteristic of individuals in the animal species, hence more likely to have enhance stability.

In some embodiment of the invention, a library of human antibody genes, particularly a library of human germline antibody genes is used to identify somatic hypermutation in a given antibody resulting from an immunization campaign. For example, germline DNA and the encoded protein sequences for human heavy and light chain variable domain genes may be found at IMGT®, the international ImMunoGeneTics information System®, Web Resources, http://www_imgt_org.

Another embodiment of the invention is a library of antibody molecules, wherein each antibody molecule comprises a VH domain consisting of a set of VH complementarity determining regions HCDR1, HCDR2 and HCDR3, and framework regions FR1, FR2, FR3 and FR4, and a VL domain consisting of a set of VL complementarity determining regions LCDR1, LCDR2 and LCDR3, and framework regions FR1, FR2, FR3 and FR4, and a wherein one or more residues of the framework which had undergone SHM has been mutated to a germline residue.

In some cases, the VH and VL framework regions of the antibody comprise one or more amino acid substitutions, deletions, and/or insertions relative to the germline amino acid sequence of the human gene. In some cases, the VH and VL framework regions of the antibody comprises 1, 2, 3, 4, 5, 6, 7, 8, 9, or 10 amino acid substitutions relative to the germline amino acid sequence. In some cases, one or more of those substitutions, deletions, and/or insertions is in a framework region of the heavy chain and light chain. In some cases, one or more of those substitutions, deletions, and/or insertions is in a CDR of the heavy chain and light chain. In some cases, substitution may represent conservative or non-conservative amino acid substitutions at such position(s) relative to the amino acid in the reference antibody.

In some cases, the variable domain of the heavy chain comprises 1, 2, 3, 4, 5, 6, 7, 8, 9, or 10 substitutions, deletions and/or insertions from the germline amino acid sequence. In some cases, the substitution is a non-conservative substitution compared to the germline amino acid sequence. In some cases, the substitution, deletion and/or insertion are in a framework region of the heavy chain. In some cases, the amino acid substitution, deletion and/or are in the CDR regions of the heavy chain.

In some cases, the variable domain of the light chain comprises 1, 2, 3, 4, 5, 6, 7, 8, 9, or 10 substitutions, deletions and/or insertions from the germline amino acid sequence. In some cases, the substitution is a non-conservative substitution compared to the germline amino acid sequence. In some cases, the substitution, deletion and/or insertion are in a framework region of the light chain. In some cases, the amino acid substitution, deletion and/or insertion are in the CDR regions of the light chain.

In another aspect, the framework region is mutated so that the resulting framework region(s) have the amino acid sequence of the corresponding germline gene. A mutation may be made in a framework region or CDR region to increase the thermal stability of the antibody and improve shelf-life. A mutation in a framework region can also be made to alter or reduce the immunogenicity of the antibody, A single antibody may have mutations in any one or more of the CDRs or framework regions of the variable domain or in the constant domain.

The term “germlining” is the process of reversing one or more amino acid found in an antibody VH or VL sequence to the corresponding amino acid of a germline sequence. In some examples, germlining involves replacing an unusual or low frequency residue with an equivalent residue from the closest matching germline sequence. The germlining of a VH or VL domain having an amino acid sequence homologous to a member of the human VH3 family will often involve replacement/substitution of a residue which was found to be an unusual or low frequency residue or rare residue at that position. The unusual or low frequency residue may be the result of a somatic hypermutation.

The general principles of germlining described herein apply equally in this embodiment of the invention. By way of example, lead selected clones containing unusual or low frequency residues in the VH and VL domains may be germlined in their framework regions (FRs) by applying a library approach. After alignment against the closest human germline (for VH and VL) and other human germlines, the residues to be changed in the FRs are identified and the human residue is selected. Whilst germlining may involve replacement of somatic hypermutation residues with an equivalent residue from the closest matching human germline this is not essential, and residues from other human germlines could also be used.

The overall aim of the germlining process is to produce a molecule in which the VH and VL domains exhibit minimal immunogenicity when introduced into a human subject and improved stability, whilst retaining the specificity and affinity of the antigen binding site formed by the parental VH and VL domains

“Unusual residues” refers to amino acid residues found in the variable regions of an antibody whose frequency is less than 1% compared to the frequency of the amino acid residue found in the germline antibody.

“Low frequency residues” refers to amino acid residues found in the variable regions of an antibody whose frequency is low compared to the frequency of the amino acid residue found in the germline antibody. The frequency may be a frequency of 1%, 2%, 3%, 4%, 5%, 6%, 7%, 8%, 9%, or 10%.

The term “K_{D}” refers to the “equilibrium dissociation constant” and refers to the value obtained in a titration measurement at equilibrium, or by dividing the dissociation rate constant (K_{off}) by the association rate constant (Kₒₙ). “Kₐ” refers to the affinity constant. The association rate constant, the dissociation rate constant and the equilibrium dissociation constant are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used.

The numbering of amino acid residues in the antibody constant region throughout the specification is according to the EU index as described in Kabat et al. (1991, J Immunol 147(5): 1709-19), unless otherwise explicitly stated.

Conventional one and three-letter amino acid codes are used herein as shown in Table 1.

TABLE 1

| Amino acid | Three-letter | One-letter code |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

### Method of Improving Antibody Stability

The invention provides a method of improving an antibody, which comprises one or more or all of the steps of identifying an antibody for optimization, identifying one or more unusual or low frequency residues in the antibody VH or VL or VH and VL; aligning the antibody VH and VL sequences with the closest human or non-human germline sequences; identifying somatic hypermutation sites in the framework of the VH and VL; identifying one or more germline residue typically observed at the site of the somatic hypermutation; designing and engineering variants or library of variants containing one or more germline mutations at the site of the somatic hypermutation; cloning and producing engineered variants; and assessing biophysical properties of the engineered variants, assessing immunogenicity risks of the engineered variants and selecting one or more optimal variants.

In certain embodiment of the invention, identification of unusual or low frequency residues is done by a computer-based software. In a particular embodiment, the computer-based software is the software abYsis. The framework sequences that may be used to identify unusual or low frequency residues may be obtained from public databases or published references that include germline antibody gene sequences. For example, germline DNA and the encoded protein sequences for human heavy and light chain variable domain genes may be found at IMGT®.

An antibody containing framework regions derived from a germline sequence refers to an antibody obtained from a system that uses human germline immunoglobulin genes, such as from transgenic mice, rats or chicken or from phage display libraries. Such antibody may contain amino acid differences as compared to the sequence it was derived from, due to, for example, naturally-occurring somatic mutations or intentional substitutions. In certain embodiment, unusual or low frequency residues of the lead antibody are somatic hypermutations in the framework regions, CDR1, CDR2 or CDR3.

Unusual or low frequency residues that may be replaced to improve stability may be those with the lowest frequency as calculated by the software abYsis (Swindells, M. B. et al. abYsis: Integrated Antibody Sequence and Structure-Management, Analysis, and Prediction. J Mol Biol 429, 356-364 (2017)). In a certain embodiment of the invention, the frequency of the unusual residues that will be replaced by a germline residue is less than 1%. The frequency of low frequency residue that will be replaced by a germline residue is 1%, 2%, 3%, 4%, 5%, 6%, 7%, 8%, 9%, or 10%.

The invention provides a method of designing variant antibodies, wherein the VH, the VL or both the VH and the VL optionally comprise one, two, three, four, five, six, seven, eight, nine, ten, eleven, twelve, thirteen, fourteen or fifteen amino acid substitutions in the framework region of the antibody. Optionally, substitutions may be within the CDR1, CDR2 or CDR3 but should not affect binding of the antibody. In the context of the invention, the substitutions are germline substitutions at sites unusual or low frequency residues were observed. Possible sites of substitution are within the framework regions of the antibody. Exemplary substitutions may be germline substitutions.

The method also comprises a step of assessing the stability of the original lead antibody and the engineered variants. In the context of the invention, stability of the antibody can be measured using any of the suitable assay known in the art, such as, for example, but not limited to, measuring the free energy of unfolding, thermal stability, quantitative size distribution of monomers and other higher order aggregates, storage, and non-specific binding. Methods of measuring protein stability include but are not limited to, analytical ultracentrifugation, differential scanning calorimetry, analytical size exclusion, differential scanning fluorimetry. Methods of predicting stability may include molecular modeling. Other methods of measuring protein stability in vivo and in vitro can also be used in the context of the invention. The stability of the antibody can be measured in terms of the transition mid-point value Tm, temperature of aggregation Tagg, free energy of unfolding ΔGu and C₅₀, change in the state of aggregation, or binding to non-specific surfaces. The term “stability” as used herein refers to the ability of an antibody to retain its structural conformation and/or its activity and/or affinity when subjected to high or low temperature, immunoglobulin aggregation, and other stresses tested in antibody manufacturing. An antibody variant with improved stability refers to an antibody variant with increase tolerance to high or low temperature, immunoglobulin aggregation, and other stresses tested during antibody manufacturing.

In some embodiments, the analytical ultracentrifugation assessment further comprises comparing the Analytical Ultracentrifugation Sedimentation Velocity (AUC-SV) of the engineered variants, to the AUC-SV of said lead antibody. Preferably, the method of the invention identifying unusual or low frequency residues in the framework region of an antibody and substituting the unusual or low frequency residues with germline residues, provides antibody variants with improved AUC-SV values. For example, the variant antibodies will exhibit >95% monomer (e.g. 95%, 96%, 97%, 98%, 99% or 100% monomer). An antibody with improved AUC-SV values will show an increase in monomer content by 2% or higher.

In some embodiments, the thermal stability assessment further comprises comparing the Tm of the thermal unfolding curve of each said engineered variant, to the Tm of the thermal unfolding curve of said lead antibody. Preferably, the method of the invention identifying unusual or low frequency residues in the framework region of an antibody and substituting the unusual or low frequency residues with germline residues, provides antibody variants with increased Tm values. The effect of one or more mutations on the thermal stability of variant antibodies as described in the invention, is determined by measuring changes in Tm values extrapolated from a thermal unfolding curve. A favorable mutation increasing the stability of the variant antibodies is expected to increase the Tm. For example, the variant antibodies will exhibit ΔTm increase of 1° C. or more (such as ΔTm of 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C.) when compared to the original lead antibody Tm.

In some embodiments, thermal stability assessment further comprises comparing the Tagg value of each said engineered variant, to the Tagg value of said lead antibody. Preferably, the method of the invention identifying unusual or low frequency residues in the framework region of an antibody and substituting the unusual or low frequency residues with germline residues, provides antibody variants with increased Tagg values. A favorable mutation increasing the stability of the variant antibodies is expected to increase the Tagg. For example, the variant antibodies will exhibit a ΔTagg of 1-25° C. (e.g. ΔTagg of 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C.) when compared to the original lead antibody Tagg.

In one embodiment, the free energy of unfolding further comprises comparing the ΔGu1, ΔGu2, C₅₀ of each engineered variant, to the ΔGu1, ΔGu2, C₅₀ of the lead antibody. Preferably, the method of the invention identifying unusual or low frequency residues in the framework region of an antibody and substituting the unusual or low frequency residues with germline residues, provides antibody variants with increased ΔGu1, ΔGu2, C₅₀ values. A favorable mutation increasing the stability of the variant antibodies may increase the ΔGu1, ΔGu2, or C₅₀ values of the variant antibodies. For example, the variant antibodies will exhibit a ΔGu1 or ΔGu2 increase of at 4 kJ/mol or more when compared to the original lead antibody ΔGu1 or ΔGu2. The variant antibody may also exhibit a C₅₀ increase of 0.1 M or more when compared to the lead antibody.

In one embodiment, the storage stability of said engineered variant is measured at 4° C., or 40° C. at 2 weeks and 4 weeks and compared to the storage stability of said lead antibody. In a particular embodiment, the storage stability is measured by looking at the change in aggregation level between time zero and 1 month. Preferably, the method of the invention identifying unusual or low frequency residues in the framework region of an antibody and substituting the unusual or low frequency residues with germline residues, provides antibody variants with reduced aggregation. For example, the variant antibodies will exhibit a decrease in Δ % aggregation of 1-5%. (e.g. Δ % Aggregation of 1, 2, 3, 4 or 5%) when compared to the original lead antibody aggregation level.

In another embodiment, the method of producing stable antibodies comprises assessing the immunogenicity risk of the engineered variant.

In certain embodiments of the invention, the immunogenicity risk assessment is measured in silico. In a particular embodiment, the immunogenicity risk assessment in silico is measured by Epivax score. In certain embodiments, the immunogenicity risk of the variant antibody is equal or lower than the immunogenicity risk of the original lead antibody.

In certain embodiments, the engineered variants are made in the human framework regions, CDR1, CDR2 or CDR3 of the antibody. The amino acid replacements can occur by any suitable method known in the art.

In some embodiments, the method of the claimed invention comprises measuring the affinity of the lead antibody and the antibody variants and comparing the affinity of the antibody variant to the affinity of the lead antibody. The affinity of the lead antibody and antibody variants may be determined experimentally using any suitable method. An exemplary method utilizes ProteOn XPR36, BIAcore 3000, Octet, KinExA instrumentation, ELISA or competitive binding assays known to those skilled in the art. The measured affinity of an antibody may vary if measured under different conditions (e.g., osmolarity, pH). Thus, measurements of affinity and other binding parameters (e.g., K_{D}, Kₒₙ, and K_{off}) are typically made with standardized conditions and a standardized buffer, such as the buffer described herein. Skilled in the art will appreciate that the internal error for affinity measurements for example using BIAcore 3000 or ProteOn (measured as standard deviation, SD) can typically be within 5-33% for measurements within the typical limits of detection. Therefore, the term “about” when referring to a K_{D} value reflects the typical standard deviation in the assay.

The method of the invention also comprises selecting variant antibodies which exhibit enhanced stability but retained affinities similar to the lead molecule. In some embodiments the affinity of the variant antibodies is functionally the same or similar, as would be understood by one having ordinary skill in the art. In other embodiment, the affinity of the variant antibody may be tighter than the affinity of the original lead antibody. At a minimum, such references that include a numerical parameter would include variations that, using mathematical and industrial principles accepted in the art (e.g., rounding, measurement or other systematic errors, manufacturing tolerances, etc.), would not vary the least significant digit.

### Example 1

The example below describes the optimization of an anti-prostate target antibody, TMEB675, through germlining of SHM sites identified in the framework of the antibody. While the antibody met the functional criteria characteristic of a high affinity antibody, it showed poor intrinsic properties. Re-engineering of TMEB675 generated a panel of variants from which TMEB762 was selected based both on its function and favorable biophysical properties.

### Discovery, Engineering and Germline Optimization

The monoclonal antibody (TMEB675) was discovered by immunizing OmniRats with the recombinant human TMEFF2 in the OmniRat® transgenic platform. OmniRat® is a therapeutic human antibody platform producing highly diversified, fully human antibody repertoires. The OmniRat® contains a chimeric human/rat IgH locus (comprising 22 human VHs, all human D and JH segments in natural configuration linked to the rat CH locus) together with fully human IgL loci (12 Vκs linked to Jκ-Cκ and 16 Vλs linked to Jλ-Cλ) (Osborn, M. J. et al. High-affinity IgG antibodies develop naturally in Ig-knockout rats carrying germline human IgH/Igkappa/Iglambda loci bearing the rat CH region. J Immunol 190, 1481-1490 (2013)). Accordingly, the rats exhibit reduced expression of rat immunoglobulin, and in response to immunization, the introduced human heavy and light chain transgenes undergo class switching and somatic mutation to generate high affinity chimeric human/rat IgG monoclonal antibodies with fully human variable regions. The preparation and use of OmniRat®, and the genomic modifications carried by such rats, is described in WO14/093908. Following a 89-day immunization regimen, lymph nodes from the rats were harvested and used to generate hybridomas. Hybridoma supernatants were screened for binding to recombinant human TMEFF2 by ELISA.

Based on the screening results, several hybridoma clones were sequenced, expressed and characterized for functionality. TMEB675 showed desirable recombinant protein affinity and cell binding attributes (Table 2) and was selected for further studies.

TABLE 2
Parameters from Affinity measurement done by SPR are provided in
this table. Association constant kₐ (M⁻¹ s⁻¹), dissociation constant k_{d}
((s⁻¹) and equilibrium constant K_{D} (M) are included.

| | kₐ | k_{d} | K_{D} |
|---|---|---|---|
| mAb | (M⁻¹ s⁻¹) | (s⁻¹) | (pM) |
| TMEB675 | 6.24 × 10⁴ | 3.16 × 10⁻⁵ | 25.7 |

The abYsis tool allows searching for “unusual residues” within the antibody heavy and light chain sequence (Swindells, M. B. et al. abYsis: Integrated Antibody Sequence and Structure-Management, Analysis, and Prediction. J Mol Biol 429, 356-364 (2017)). Unusual residues defined by a threshold of less than 1% in the database of antibody sequences offers hints about the critical functions of certain positions. Low frequency unusual residues defined by a 1-10% threshold in the database of antibody sequences offers hints about the critical functions of certain positions.

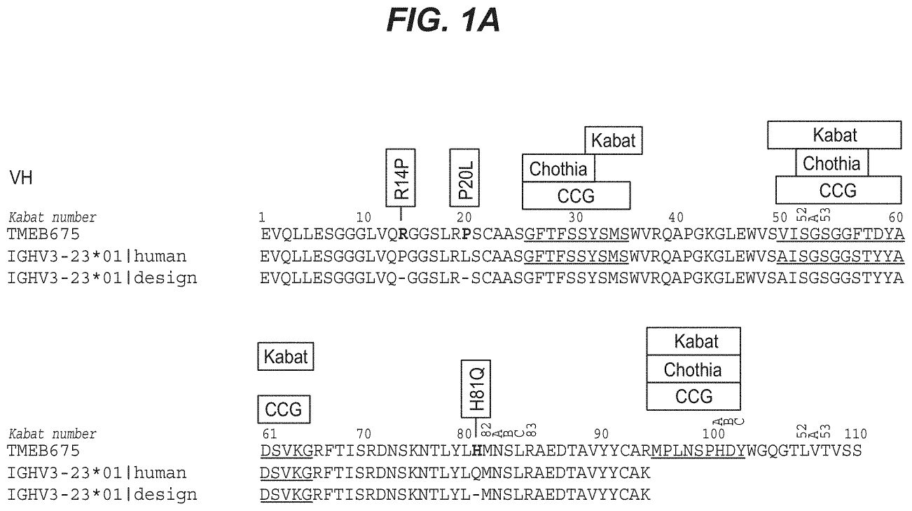

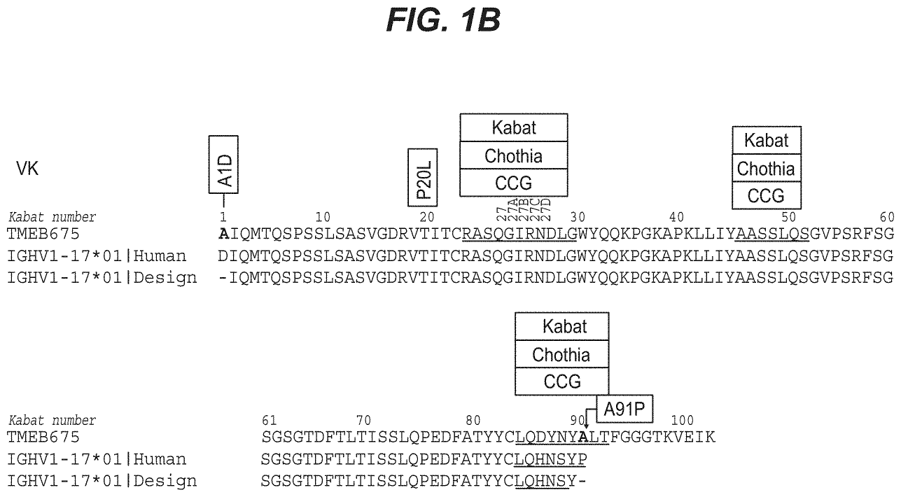

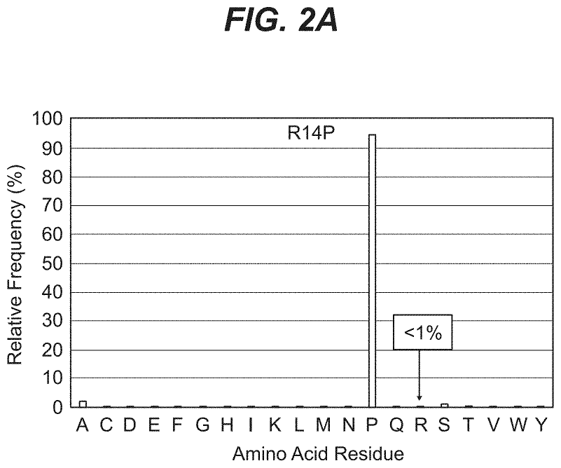

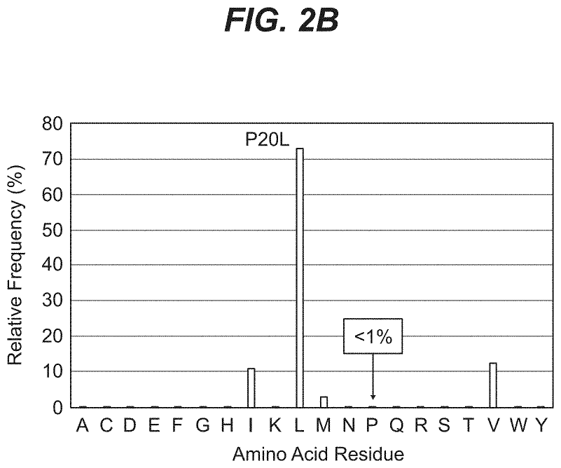

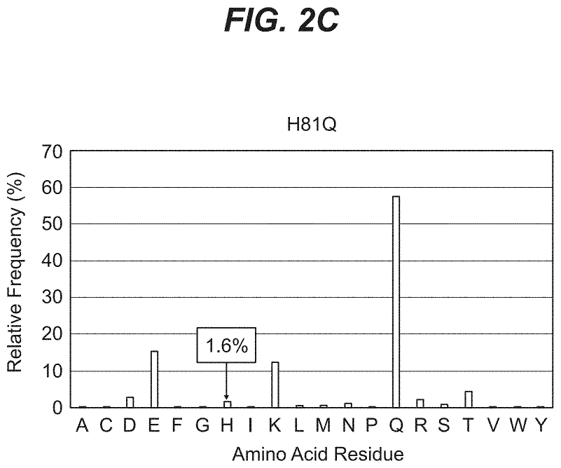

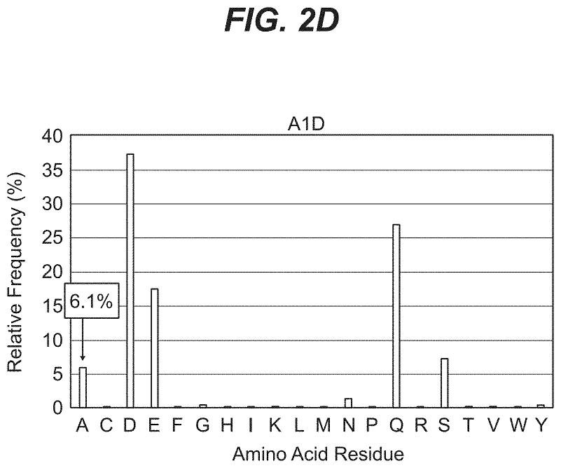

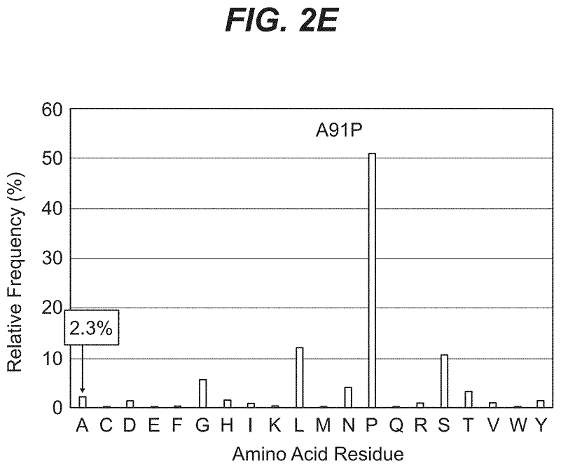

Sequence alignment of the variable heavy and light chain regions of TMEB675 with human germline sequences for VH and VL using the abYsis portal indicated several somatic hypermutations (SHM) within the framework region. Three somatic hypermutations were observed in VH (R14P, P20L, H81Q) and two SHM (A1D, A91P) were observed in Vk (FIG. 1A, FIG. 1B, FIG. 2A-D). Arginine, Proline, and Histidine amino acid residues found at position 14, 20 and 81, respectively of the heavy chain human framework (HCFR) and the Alanine residue found at position 1 of the light chain human framework, scored low frequency in the abYsis analysis, indicating that these are unusual or low frequency residues (FIG. 2A-E). The Arginine at position 14 and Proline at position 20 of HCFR of TMEB675 are low frequency residues (<1%, Table 3, 4 and 5).

The SHM Arginine found at position 14 is rare (frequency 0.151%) compared to the most frequently found Proline residue at this position (Proline residue frequency 95.029%). (Table 3 and 4, FIG. 2A). The frequency of the SHM, Proline, found at position 20 of the heavy chain is also low (frequency 0.088%) compared to most frequently found Leucine residue (frequency 73.024%). (Table 3 and 5, FIG. 2B). The frequency of finding the third SHM, Histidine at position 81 is relatively rare too (frequency 1.604%) compared to the most frequently found Glutamine residue typically found at that position (frequency 57.576%) (Table 3 and 6, FIG. 2C). Table 3 shows human heavy chain germline sequences and typical compositions at position 14, 20 and 81. Table 4, 5 and 6 show abYsis database heavy chain sequences and composition of residue at position 14, 20 and 81, respectively. Table 7 and 8 show abYsis database light chain sequences and composition of residue at position 1 and 91, respectively.

TABLE 3
Human heavy chain (IGHV) germline sequences and composition
of residues at position 14, 20 and 81.

| | | SEQ | | | | |
|---|---|---|---|---|---|---|
| | | ID | | | | |
| Germline | Allele | NO | Sequence | Pos14 | Pos20 | Pos81 |
| IGHV1-2 | IGHV1- | 1 | QVQLVQSGAEVKKPGASVKV | P | V | E |
| | 2*01 | | SCKASGYTFTGYYMHWVRQ | | | |
| | | | APGQGLEWMGRINPNSGGTN | | | |
| | | | YAQKFQGRVTSTRDTSISTAY | | | |
| | | | MELSRLRSDDTVVYYCAR | | | |
| IGHV1-3 | IGHV1- | 2 | QVQLVQSGAEVKKPGASVKV | P | V | E |
| | 3*01 | | SCKASGYTFTSYAMTIWVRQ | | | |
| | | | APGQRLEWMGWINAGNGNT | | | |
| | | | KYSQKFQGRVTITRDTSASTA | | | |
| | | | YMELSSLRSEDTAVYYCAR | | | |
| IGHV1-8 | IGHV1- | 3 | QVQLVQSGAEVKKPGASVKV | P | V | E |
| | 8*01 | | SCKASGYTFTSYDINWVRQA | | | |
| | | | TGQGLEWMGWMNPNSGNTG | | | |
| | | | YAQKFQGRVTMTRNTSISTA | | | |
| | | | YMELSSLRSEDTAVYYCAR | | | |
| IGHV1- | IGHV1- | 4 | QVQLVQSGAEVKKPGASVKV | P | V | E |
| 18 | 18*01 | | SCKASGYTFTSYGISWVRQAP | | | |
| | | | GQGLEWMGWISAYNGNTNY | | | |
| | | | AQKLQGRVTMTTDTSTSTAY | | | |
| | | | MELRSLRSDDTAVYYCAR | | | |
| IGHV1- | IGHV1- | 5 | QVQLVQSGAEVKKPGASVKV | P | V | E |
| 24 | 24*01 | | SCKVSGYTLTELSMHWVRQA | | | |
| | | | PGKGLEWMGGFDPEDGETIY | | | |
| | | | AQKFQGRVTMTEDTSTDTAY | | | |
| | | | MELSSLRSEDTAVYYCAT | | | |
| IGHV1- | IGHV1- | 6 | QVQLVQSWAEVRKSGASVK | S | V | D |
| 38-4 | 38-4*01 | | VSCSFSGFTITSYGIHWVQQS | | | |
| | | | PGQGLEWMGWINPGNGSPSY | | | |
| | | | AKKFQGRFTMTRDMSTTTAY | | | |
| | | | TDLSSLTSEDMAVYYYAR | | | |
| IGHV1- | IGHV1- | 7 | QMQLVQSGAEVKKTGSSVK | T | V | E |
| 45 | 45*01 | | VSCKASGYTFTYRYLHWVRQ | | | |
| | | | APGQALEWMGWITPFNGNTN | | | |
| | | | YAQKFQDRVTITRDRSMSTA | | | |
| | | | YMELSSLRSEDTAMYYCAR | | | |
| IGHV1- | IGHV1- | 8 | QVQLVQSGAEVKKPGASVKV | P | V | E |
| 46 | 46*01 | | SCKASGYTFTSYYMFIWVRQ | | | |
| | | | APGQGLEWMGIINPSGGSTSY | | | |
| | | | AQKFQGRVTMTRDTSTSTVY | | | |
| | | | MELSSLRSEDTAVYYCAR | | | |
| IGHV1- | IGHV1- | 9 | QMQLVQSGPEVKKPGTSVKV | P | V | E |
| 58 | 58*01 | | SCKASGFTFTSSAVQWVRQA | | | |
| | | | RGQRLEWIGWIVVGSGNTNY | | | |
| | | | AQKFQERVTITRDMSTSTAY | | | |
| | | | MELSSLRSEDTAVYYCAA | | | |
| IGHV1- | IGHV1- | 10 | QVQLGQSEAEVKKPGASVKV | P | V | E |
| 68 | 68*01 | | SCKASGYTFTCCSLHWLQQA | | | |
| | | | PGQGLERMRWITLYNGNTNY | | | |
| | | | AKKFQGRVTITRDMSLRTAYI | | | |
| | | | ELSSLRSEDSAVYYWAR | | | |
| IGHV1- | IGHV1- | 11 | QVQLVQSGAEVKKPGSSVKV | P | V | E |
| 69 | 69*01 | | SCKASGGTFSSYAISWVRQAP | | | |
| | | | GQ GLEWMGGIIPIFGTANYAQ | | | |
| | | | KFQGRVTITADESTSTAYMEL | | | |
| | | | SSLRSEDTAVYYCAR | | | |
| IGHV1- | IGHV1- | 12 | EVQLVQSGAEVKKPGATVKI | P | I | E |
| 69-2 | 69-2*01 | | SCKVSGYTFTDYYMHWVQQ | | | |
| | | | APGKGLEWMGLVDPEDGETI | | | |
| | | | YAEKFQGRVTITADTSTDTAY | | | |
| | | | MELSSLRSEDTAVYYC AT | | | |
| IGHV1- | IGHV1- | 13 | QVQLVQSGAEVKKPGSSVKV | P | V | E |
| 69D | 69D*01 | | SCKASGGTFSSYAISWVRQAP | | | |
| | | | GQ GLEWMGGIIPIFGTANYAQ | | | |
| | | | KFQGRVTITADESTSTAYMEL | | | |
| | | | SSLRSEDTAVYYCAR | | | |
| IGHV1- | IGHV1- | 14 | QVQLLQPGVQVKKPGSSVKV | P | V | E |
| NL1 | NL1*01 | | SCKASRYTFTKYFTRWVRQS | | | |
| | | | PGQGHXWMGWINPYNDNTH | | | |
| | | | YAQTFWGRVTITSDRSMSTA | | | |
| | | | YMELSXLRSEDMVVYYCVR | | | |
| IGHV2-5 | IGHV2- | 15 | QITLKESGPTLVKPTQTLTLTC | P | L | T |
| | 5*01 | | TFSGFSLSTSGVGVGWIRQPP | | | |
| | | | GKALEWLALIYWNDDKRYSP | | | |
| | | | SLKSRLTITKDTSKNQVVLTM | | | |
| | | | TNMDPVDTATYYCAHR | | | |
| IGHV2- | IGHV2- | 16 | QVTLKESGPALVKPTQTLML | P | L | T |
| 10 | 10*01 | | TCTFSGFSLSTSGMGVGWICQ | | | |
| | | | PSAKALEWLAHIYWNDNKY | | | |
| | | | YSPSLKSRLIISKDTSKNEVVL | | | |
| | | | TVINMDIVDTATHYCARR | | | |
| IGHV2- | IGHV2- | 17 | QVTLKESGPVLVKPTETLTLT | P | L | T |
| 26 | 26*01 | | CTVSGFSLSNARMGVSWIRQ | | | |
| | | | PPGKALEWLAHIFSNDEKSYS | | | |
| | | | TSLKSRLTISKDTSKSQVVLT | | | |
| | | | MTNMDPVDTATYYCARI | | | |
| IGHV2- | IGHV2- | 18 | QVTLRESGPALVKPTQTLTLT | P | L | T |
| 70 | 70*01 | | CTFSGFSLSTSGMCVSWIRQP | | | |
| | | | PGKALEWLALIDWDDDKYYS | | | |
| | | | TSLKTRLTISKDTSKNQVVLT | | | |
| | | | MTNMDPVDTATYYCARI | | | |
| IGHV3-7 | IGHV3- | 19 | EVQLVESGGGLVQPGGSLRL | P | L | Q |
| | 7*01 | | SCAASGFTFSSYWMSWVRQA | | | |
| | | | PGKGLEWVANIKQDGSEKYY | | | |
| | | | VDSVKGRFTISRDNAKNSLYL | | | |
| | | | QMNSLRAEDTAVYYCAR | | | |
| IGHV3-9 | IGHV3- | 20 | EVQLVESGGGLVQPGRSLRLS | P | L | Q |
| | 9*01 | | CAASGFTFDDYAMHWVRQA | | | |
| | | | PGKGLEWVSGISWNSGSIGY | | | |
| | | | ADSVKGRFTISRDNAKNSLYL | | | |
| | | | QMNSLRAEDTALYYCAKD | | | |
| IGHV3- | IGHV3- | 21 | QVQLVESGGGLVKPGGSLRL | P | L | Q |
| 11 | 11*01 | | SCAASGFTFSDYYMSWIRQA | | | |
| | | | PGKGLEWVSYISSSGSTIYYA | | | |
| | | | DSVKGRFTISRDNAKNSLYLQ | | | |
| | | | MNSLRAEDTAVYYCAR | | | |
| IGHV3- | IGHV3- | 22 | VQLVESGGGLVQPGGSLRLS | P | L | Q |
| 13 | 13*01 | | CAASGFTFSSYDMHWVRQAT | | | |
| | | | GKGLEWVSAIGTAGDTYYPG | | | |
| | | | SVKGRFTISRENAKNSLYLQM | | | |
| | | | NSLRAGDTAVYYCAR | | | |
| IGHV3- | IGHV3- | 23 | EVQLVESGGGLVKPGGSLRL | P | L | Q |
| 15 | 15*01 | | SCAASGFTFSNAWMSWVRQ | | | |
| | | | APGKGLEWVGRIKSKTDGGT | | | |
| | | | TDYAAPVKGRFTISRDDSKNT | | | |
| | | | LYLQMNSLKTEDTAVYYCTT | | | |
| IGHV3- | IGHV3- | 24 | EVQLVESGGGLVQPGGSLRL | P | L | Q |
| 16 | 16*01 | | SCAASGFTFSNSDMNWARKA | | | |
| | | | PGKGLEWVSGVSWNGSRTH | | | |
| | | | YVDSVKRRFIISRDNSRNSLY | | | |
| | | | LQKNRRRAEDMAVYYCVR | | | |
| IGHV3- | IGHV3- | 25 | TVQLVESGGGLVEPGGSLRLS | P | L | Q |
| 19 | 19*01 | | CAASGFTFSNSDMNWVRQAP | | | |
| | | | GKGLEWVSGVSWNGSRTHY | | | |
| | | | ADSVKGRFIISRDNSRNFLYQ | | | |
| | | | QMNSLRPEDMAVYYCVR | | | |
| IGHV3- | IGHV3- | 26 | EVQLVESGGGVVRPGGSLRL | P | L | Q |
| 20 | 20*01 | | SCAASGFTFDDYGMSWVRQ | | | |
| | | | APGKGLEWVSGINWNGGSTG | | | |
| | | | YADSVKGRFTISRDNAKNSL | | | |
| | | | YLQMNSLRAEDTALYHCAR | | | |
| IGHV3- | IGHV3- | 27 | EVQLVESGGGLVKPGGSLRL | P | L | Q |
| 21 | 21*01 | | SCAASGFTFSSYSMNWVRQA | | | |
| | | | PGKGLEWVSSISSSSSYIYYA | | | |
| | | | DSVKGRFTISRDNAKNSLYLQ | | | |
| | | | MNSLRAEDTAVYYCAR | | | |
| IGHV3- | IGHV3- | 28 | EVHLVESGGALVQPGGSLRL | P | L | Q |
| 22 | 22*01 | | SCAASGFTFSYYYMSGVRQA | | | |
| | | | PGKGLEWVGFIRNKANGGTT | | | |
| | | | EYTTSVKGRFTISRDDSKSITY | | | |
| | | | LQMKSLKTEDTAVYYCSR | | | |
| IGHV3- | IGHV3- | 29 | EVQLLESGGGLVQPGGSLRLS | P | L | Q |
| 23 | 23*01 | | CAASGFTFSSYAMSWVRQAP | | | |
| | | | GKGLEWVSAISGSGGSTYYA | | | |
| | | | DSVKGRFTISRDNSKNTLYLQ | | | |
| | | | MNSLRAEDTAVYYCAK | | | |
| IGHV3- | IGHV3- | 30 | EVQLLESGGGLVQPGGSLRLS | P | L | Q |
| 23D | 23D*01 | | CAASGFTFSSYAMSWVRQAP | | | |
| | | | GKGLEWVSAISGSGGSTYYA | | | |
| | | | DSVKGRFTISRDNSKNTLYLQ | | | |
| | | | MNSLRAEDTAVYYCAK | | | |
| IGHV3- | IGHV3- | 31 | EMQLVESGGGLQKPAWSPRL | P | L | Q |
| 25 | 25*01 | | SCAASQFTFSSYYMNCVRQA | | | |
| | | | PGNGLELVSQVNPNGGSTYLI | | | |
| | | | DSGKDRFNTSRDNAKNTLHL | | | |
| | | | QMNSLKTEDTALYYCTR | | | |
| IGHV3- | IGHV3- | 32 | EVELIEPTEDLRQPGKFLRLSC | P | L | Q |
| 29 | 29*01 | | VASRFAFSSFWMSPVHQSAG | | | |
| | | | KGLEWVIDIKDDGSQIHHADS | | | |
| | | | VKGRFSISKDNAKNSLYLQM | | | |
| | | | NSQRTEDMAVYGCT | | | |
| IGHV3- | IGHV3- | 33 | QVQLVESGGGVVQPGRSLRL | P | L | Q |
| 30 | 30*01 | | SCAASGFTFSSYAMHWVRQA | | | |
| | | | PGKGLEWVAVISYDGSNKYY | | | |
| | | | ADSVKGRFTISRDNSKNTLYL | | | |
| | | | QMNSLRAEDTAVYYCAR | | | |
| IGHV3- | IGHV3- | 34 | EVQLVESGEDPRQPGGSLRLS | P | L | L |
| 30-2 | 30-2*01 | | CADSGLTFSSYARNSVSQAPG | | | |
| | | | KGLEWVVDIQCDGSQICYAD | | | |
| | | | SLKSKFTISKENAKNSLYLLM | | | |
| | | | NSLRAAGTAVCYCM | | | |
| IGHV3- | IGHV3- | 35 | QVQLVESGGGVVQPGRSLRL | P | L | Q |
| 30-3 | 30-3*01 | | SCAASGFTFSSYAMHWVRQA | | | |
| | | | PGKGLEWVAVISYDGSNKYY | | | |
| | | | ADSVKGRFTISRDNSKNTLYL | | | |
| | | | QMNSLRAEDTAVYYCAR | | | |
| IGHV3- | IGHV3- | 36 | QVQLVESGGGVVQPGRSLRL | P | L | Q |
| 30-5 | 30-5*01 | | SCAASGFTFSSYGMHWVRQA | | | |
| | | | PGKGLEWVAVISYDGSNKYY | | | |
| | | | ADSVKGRFTISRDNSKNTLYL | | | |
| | | | QMNSLRAEDTAVYYCAK | | | |
| IGHV3- | IGHV3- | 37 | EVELIESIEDLRQPGKFLRLSC | P | L | Q |
| 30-22 | 30- | | VASRFAFSSFGMSRVHQSPG | | | |
| | 22*01 | | KGLEWVIDIKDDGSQIHHADS | | | |
| | | | VKGRFSISKDNAKNSLYLQM | | | |
| | | | NSQRAEDMDVYGCT | | | |
| IGHV3- | IGHV3- | 38 | EVQLVESGEDPRQPGGSLRLS | P | L | L |
| 30-33 | 30- | | CADSGLTFSSYGRSSVSQAPG | | | |
| | 33*01 | | KGLEWVVDIQCDGSQICYAD | | | |
| | | | SLKSKFTISKENAKNSLYLLM | | | |
| | | | NSLRAEGTAVCYCM | | | |
| IGHV3- | IGHV3- | 39 | EVELIEPTEDLRQPGKFLRLSC | P | L | Q |
| 30 42 | 30- | | VASRFAFSSFGMSPVHQSAG | | | |
| | 42*01 | | KGLEWVIDIKDDGSQIHHADS | | | |
| | | | VKGRFSISKDNAKNSLYLQM | | | |
| | | | NSQRTEDMAVYGCT | | | |
| IGHV3- | IGHV3- | 40 | EVQLVESGEDPRQPGGSLRLS | P | L | L |
| 30-52 | 30- | | CADSGLTFSSYGRNSVSQAPG | | | |
| | 52*01 | | KGLEWVVDIQCDGSQICYAD | | | |
| | | | SLKSKFTISKENAKNSLYLLM | | | |
| | | | NSLRAAGTAVCYCM | | | |
| IGHV3- | IGHV3- | 41 | EVELIESIEDLRQPGKFLRLSC | P | L | Q |
| 32 | 32*01 | | VASRFAFSSFGMSRVHQSPG | | | |
| | | | KGLEWVIDIKDDGSQIHHADS | | | |
| | | | VKGRFSISKDNAKNSLYLQM | | | |
| | | | NTQRAEDVAVYGYT | | | |
| IGHV3- | IGHV3- | 42 | QVQLVESGGGVVQPGRSLRL | P | L | Q |
| 33 | 33*01 | | SCAASGFTFSSYGMHWVRQA | | | |
| | | | PGKGLEWVAVIWYDGSNKY | | | |
| | | | YADSVKGRFTISRDNSKNTLY | | | |
| | | | LQMNSLRAEDTAVYYCAR | | | |
| IGHV3- | IGHV3- | 43 | EVQLVESGEDPRQPGGSLRLS | P | L | Q |
| 33-2 | 33-2*01 | | CADSGLTFSSYGMSSVSQAP | | | |
| | | | GKGLEWVVDIQCDGSQICYA | | | |
| | | | QSVKSKFTISKENAKNSLYLQ | | | |
| | | | MNSLRAEGTAVCYCM | | | |
| IGHV3- | IGHV3- | 44 | EVQLVESGGGLVQPGGSLRL | P | L | Q |
| 35 | 35*01 | | SCAASGFTFSNSDMNWVHQA | | | |
| | | | PGKGLEWVSGVSWNGSRTH | | | |
| | | | YADSVKGRFIISRDNSRNTLY | | | |
| | | | LQTNSLRAEDTAVYYCVR | | | |
| IGHV3- | IGHV3- | 45 | EVQLVESGGGLVQPRGSLRLS | P | L | Q |
| 38 | 38*01 | | CAASGFTVSSNEMSWIRQAP | | | |
| | | | GKGLEWVSSISGGSTYYADS | | | |
| | | | RKGRFTISRDNSKNTLYLQM | | | |
| | | | NNLRAEGTAAYYCARY | | | |
| IGHV3- | IGHV3- | 46 | EVQLVESRGVLVQPGGSLRLS | P | L | Q |
| 38-3 | 38-3*01 | | CAASGFTVSSNEMSWVRQAP | | | |
| | | | GKGLEWVSSISGGSTYYADS | | | |
| | | | RKGRFTISRDNSKNTLHLQM | | | |
| | | | NSLRAEDTAVYYCKK | | | |
| IGHV3- | IGHV3- | 47 | EVQLVESGGVVVQPGGSLRL | P | L | Q |
| 43 | 43*01 | | SCAASGFTFDDYTMHWVRQ | | | |
| | | | APGKGLEWVSLISWDGGSTY | | | |
| | | | YADSVKGRFTISRDNSKNSLY | | | |
| | | | LQMNSLRTEDTALYYCAKD | | | |
| IGHV3- | IGHV3- | 48 | EDQLVESGGGLVQPGGSLRPS | P | P | H |
| 47 | 47*01 | | CAASGFAFSSYALHWVRRAP | | | |
| | | | GKGLEWVSAIGTGGDTYYAD | | | |
| | | | SVMGRFTISRDNAKKSLYLH | | | |
| | | | MNSLIAEDMAVYYCAR | | | |
| IGHV3- | IGHV3- | 49 | EVQLVESGGGLVQPGGSLRL | P | L | Q |
| 48 | 48*01 | | SCAASGFTFSSYSMNWVRQA | | | |
| | | | PGKGLEWVSYISSSSSTIYYA | | | |
| | | | DSVKGRFTISRDNAKNSLYLQ | | | |
| | | | MNSLRAEDTAVYYCAR | | | |
| IGHV3- | IGHV3- | 50 | EVQLVESGGGLVQPGRSLRLS | P | L | Q |
| 49 | 49*01 | | CTASGFTFGDYAMSWFRQAP | | | |
| | | | GKGLEWVGFIRSKAYGGTTE | | | |
| | | | YTASVKGRFTISRDGSKSIAY | | | |
| | | | LQMNSLKTEDTAVYYCTR | | | |
| IGHV3- | IGHV3- | 51 | EVQLVESGGGLVQPGGSLRL | P | L | Q |
| 52 | 52*01 | | SCAASGFTFSSSWMEIWVCQA | | | |
| | | | PEKGLEWVADIKCDGSEKYY | | | |
| | | | VDSVKGRLTISRDNAKNSLYL | | | |
| | | | QVNSLRAEDMTVYYCVR | | | |
| IGHV3- | IGHV3- | 52 | EVQLVESGGGLIQPGGSLRLS | P | L | Q |
| 53 | 53*01 | | CAASGFTVSSNYMSWVRQAP | | | |
| | | | GKGLEWVSVIYSGGSTYYAD | | | |
| | | | SVKGRFTISRDNSKNTLYLQM | | | |
| | | | NSLRAEDTAVYYCAR | | | |
| IGHV3- | IGHV3- | 53 | EVQLVESEENQRQLGGSLRLS | L | L | Q |
| 54 | 54*01 | | CADSGLTFSSYYMSSDSQAP | | | |
| | | | GKGLEWVVDIKQDRSQLCYA | | | |
| | | | QSVKSRFTISKENAKNSLCLQ | | | |
| | | | MNSLRAEGTAVYYCM | | | |
| IGHV3- | IGHV3- | 54 | EVQLVESGEGLVQPGGSLRLS | P | L | Q |
| 62 | 62*01 | | CAASGFTFSSSAMHWVRQAP | | | |
| | | | RKGLEWVSVISTSGDTVLYT | | | |
| | | | DSVKGRFTISRDNAQNSLSLQ | | | |
| | | | MNSLRAEGTVVYYCVK | | | |
| IGHV3- | IGHV3- | 55 | EVELIESIEGLRQLGKFLRLSC | L | L | Q |
| 63 | 63*01 | | VASGFTFSSYAMSWVNETLG | | | |
| | | | KGLEGVIDVKYDGSQIYHAD | | | |
| | | | SVKGRFTISKDNAKNSPYLQT | | | |
| | | | NSLRAEDMTMEIGCT | | | |
| IGHV3- | IGHV3- | 56 | EVQLVESGGGLVQPGGSLRL | P | L | Q |
| 64 | 64*01 | | SCAASGFTFSSYAMHWVRQA | | | |
| | | | PGKGLEYVSAISSNGGSTYYA | | | |
| | | | NSVKGRFTISRDNSKNTLYLQ | | | |
| | | | MGSLRAEDMAVYYCAR | | | |
| IGHV3- | IGHV3- | 57 | EVQLVESGGGLVQPGGSLRL | P | L | Q |
| 66 | 66*01 | | SCAASGFTVSSNYMSWVRQA | | | |
| | | | PGKGLEWVSVIYSGGSTYYA | | | |
| | | | DSVKGRFTISRDNSKNTLYLQ | | | |
| | | | MNSLRAEDTAVYYCAR | | | |
| IGHV3- | IGHV3- | 58 | EVQLVESGGGLVKPGGSLRL | P | L | Q |
| 69-1 | 69-1*01 | | SCAASGFTFSDYYMNWVRQ | | | |
| | | | APGKGLEWVSSISSSSTIYYA | | | |
| | | | DSVKGRFTISRDNAKNSLYLQ | | | |
| | | | MNSLRAEDTAVYYCAR | | | |
| IGHV3- | IGHV3- | 59 | EVQLVESGGGLVQPGGSLRL | P | L | Q |
| 71 | 71*01 | | SCAASGFTFSDYYMSWVRQA | | | |
| | | | PGKGLEWVGFIRNKANGGTT | | | |
| | | | EYTTSVKGRFTISRDDSKSITY | | | |
| | | | LQMNSLRAEDTAVYYCAR | | | |
| IGHV3- | IGHV3- | 60 | EVQLVESGGGLVQPGGSLRL | P | L | Q |
| 72 | 72*01 | | SCAASGFTFSDHYMDWVRQ | | | |
| | | | APGKGLEWVGRTRNKANSY | | | |
| | | | TTEYAASVKGRFTISRDDSKN | | | |
| | | | SLYLQMNSLKTEDTAVYYCA | | | |
| | | | R | | | |
| IGHV3- | IGHV3- | 61 | EVQLVESGGGLVQPGGSLKL | P | L | Q |
| 73 | 73*01 | | SCAASGFTFSGSAMHWVRQA | | | |
| | | | SGKGLEWVGRIRSKANSYAT | | | |
| | | | AYAASVKGRFTISRDDSKNT | | | |
| | | | AYLQMNSLKTEDTAVYYCTR | | | |
| IGHV3- | IGHV3- | 62 | EVQLVESGGGLVQPGGSLRL | P | L | Q |
| 74 | 74*01 | | SCAASGFTFSSYWMFIWVRQ | | | |
| | | | APGKGLVWVSRINSDGSSTSY | | | |
| | | | ADSVKGRFTISRDNAKNTLYL | | | |
| | | | QMNSLRAEDTAVYYCAR | | | |
| IGHV3- | IGHV3- | 63 | QVQLVESGGGVVQPGGSLRL | P | L | Q |
| NL1 | NL1*01 | | SCAASGFTFSSYGMHWVRQA | | | |
| | | | PGKGLEWVSVIYSGGSSTYY | | | |
| | | | ADSVKGRFTISRDNSKNTLYL | | | |
| | | | QMNSLRAEDTAVYYCAK | | | |
| IGHV4-4 | IGHV4- | 64 | QVQLQESGPGLVKPPGTLSLT | P | L | K |
| | 4*01 | | CAVSGGSISSSNWWSWVRQP | | | |
| | | | PGKGLEWIGEIYHSGSTNYNP | | | |
| | | | SLKSRVTISVDKSKNQFSLKL | | | |
| | | | SSVTAADTAVYCCAR | | | |
| IGHV4- | IGHV4- | 65 | QVQLQESGPGLVKPSDTLSLT | P | L | K |
| 28 | 28*01 | | CAVSGYSISSSNWWGWIRQP | | | |
| | | | PGKGLEWIGYIYYSGSTYYNP | | | |
| | | | SLKSRVTMSVDTSKNQFSLKL | | | |
| | | | SSVTAVDTAVYYCAR | | | |
| IGHV4- | IGHV4- | 66 | QLQLQESGSGLVKPSQTLSLT | P | L | K |
| 30-2 | 30-2*01 | | CAVSGGSISSGGYSWSWIRQP | | | |
| | | | PGKGLEWIGYIYHSGSTYYNP | | | |
| | | | SLKSRVTISVDRSKNQFSLKL | | | |
| | | | SSVTAADTAVYYCAR | | | |
| IGHV4- | IGHV4- | 67 | QVQLQESGPGLVKPSQTLSLT | P | L | K |
| 30-4 | 30-4*01 | | CTVSGGSISSGDYYWSWIRQP | | | |
| | | | PGKGLEWIGYIYYSGSTYYNP | | | |
| | | | SLKSRVTISVDTSKNQFSLKLS | | | |
| | | | SVTAADTAVYYCAR | | | |
| IGHV4- | IGHV4- | 68 | QVQLQESGPGLVKPSQTLSLT | P | L | K |
| 31 | 31*01 | | CTVSGGSISSGGYYWSWIRQ | | | |
| | | | HPGKGLEWIGYIYYSGSTYY | | | |
| | | | NPSLKSLVTISVDTSKNQFSL | | | |
| | | | KLSSVTAADTAVYYCAR | | | |
| IGHV4- | IGHV4- | 69 | QVQLQQWGAGLLKPSETLSL | P | L | K |
| 34 | 34*01 | | TCAVYGGSFSGYYWSWIRQP | | | |
| | | | PGKGLEWIGEINHSGSTNYNP | | | |
| | | | SLKSRVTISVDTSKNQFSLKLS | | | |
| | | | SVTAADTAVYYCAR | | | |
| IGHV4- | IGHV4- | 70 | QVQLQESGPGLVKPSETLSLT | P | L | K |
| 38-2 | 38-2*01 | | CAVSGYSISSGYYWGWIRQPP | | | |
| | | | GKGLEWIGSIYHSGSTYYNPS | | | |
| | | | LKSRVTISVDTSKNQFSLKLSS | | | |
| | | | VTAADTAVYYCAR | | | |
| IGHV4- | IGHV4- | 71 | QLQLQESGPGLVKPSETLSLT | P | L | K |
| 39 | 39*01 | | CTVSGGSISSSSYYWGWIRQP | | | |
| | | | PGKGLEWIGSIYYSGSTYYNP | | | |
| | | | SLKSRVTISVDTSKNQFSLKLS | | | |
| | | | SVTAADTAVYYCAR | | | |
| IGHV4- | IGHV4- | 71 | QVQLQESGPGLVKPSETLSLI | P | L | K |
| 55 | 55*01 | | CAVSGDSISSGNWGWVRQPP | | | |
| | | | GKGLEWIGEIHHSGSTYYNPS | | | |
| | | | LKSRITMSVDTSKNQFYLKLS | | | |
| | | | SVTAADTAVYYCAR | | | |
| IGHV4- | IGHV4- | 73 | QVQLQESGPGLVKPSETLSLT | P | L | K |
| 59 | 59*01 | | CTVSGGSISSYYWSWIRQPPG | | | |
| | | | KGLEWIGYIYYSGSTNYNPSL | | | |
| | | | KSRVTISVDTSKNQFSLKLSS | | | |
| | | | VTAADTAVYYCAR | | | |
| IGHV4- | IGHV4- | 74 | QVQLQESGPGLVKPSETLSLT | P | L | K |
| 61 | 61*01 | | CTVSGGSVSSGSYYWSWIRQ | | | |
| | | | PPGKGLEWIGYIYYSGSTNYN | | | |
| | | | PSLKSRVTISVDTSKNQFSLKL | | | |
| | | | SSVTAADTAVYYCAR | | | |
| IGHV5- | IGHV5- | 75 | EVQLVQSGAEVKKPGESLRIS | P | I | Q |
| 10-1 | 10-1*01 | | CKGSGYSFTSYWISWVRQMP | | | |
| | | | GKGLEWMGRIDPSDSYTNYS | | | |
| | | | PSFQGHVTISADKSISTAYLQ | | | |
| | | | WSSLKASDTAMYYCAR | | | |
| IGHV5- | IGHV5- | 76 | EVQLVQSGAEVKKPGESLKIS | P | I | Q |
| 51 | 51*01 | | CKGSGYSFTSYWIGWVRQMP | | | |
| | | | GKGLEWMGIIYPGDSDTRYSP | | | |
| | | | SFQGQVTISADKSISTAYLQW | | | |
| | | | SSLKASDTAMYYCAR | | | |
| IGHV5- | IGHV5- | 77 | EVQLLQSAAEVKRPGESLRIS | P | I | Q |
| 78 | 78*01 | | CKTSGYSFTSYWIHWVRQMP | | | |
| | | | GKELEWMGSIYPGNSDTRYS | | | |
| | | | PSFQGHVTISADSSSSTAYLQ | | | |
| | | | WSSLKASDAAMYYCVR | | | |
| IGHV6-1 | IGHV6- | 78 | QVQLQQSGPGLVKPSQTLSLT | P | L | Q |
| | 1*01 | | CAISGDSVSSNSAAWNWIRQS | | | |
| | | | PSRGLEWLGRTYYRSKWYN | | | |
| | | | DYAVSVKSRITINPDTSKNQF | | | |
| | | | SLQLNSVTPEDTAVYYCAR | | | |
| IGHV7-4- | IGHV7- | 79 | QVQLVQSGSELKKPGASVKV | P | V | Q |
| 1 | 4-1*01 | | SCKASGYTFTSYAMNWVRQ | | | |
| | | | APGQGLEWMGWINTNTGNP | | | |
| | | | TYAQGFTGRFVFSLDTSVSTA | | | |
| | | | YLQICSLKAEDTAVYYCAR | | | |
| IGHV7- | IGHV7- | 80 | QLQLVQSGPEVKKPGASVKV | P | V | Q |
| 34-1 | 34-1*01 | | SYKSSGYTFTIYGMNWVRQT | | | |
| | | | PGQGFEWMGWIITYTGNPTY | | | |
| | | | THGFTGWFVFSMDTSVSTAC | | | |
| | | | LQISSLKAEDTAEYYCAK | | | |
| IGHV7- | IGHV7- | 81 | QVQLVQSGHEVKQPGASVKV | P | V | Q |
| 81 | 81*01 | | SCKASGYSFTTYGMNWVPQA | | | |
| | | | PGQGLEWMGWFNTYTGNPT | | | |
| | | | YAQGFTGRFVFSMDTSASTA | | | |
| | | | YLQISSLKAEDMAMYYCAR | | | |
| IGHV8- | IGHV8- | 82 | EAQLTESGGDLVHPEGPLRLS | * | L | Q |
| 51-1 | 51-1*01 | | CAASWFTFSIYEIHWVCQASG | | | |
| | | | KGLEWVAVIWRSESHQYNA | | | |
| | | | DYVRGRLTTSRDNTKYMLY | | | |
| | | | MQMNSLRTQNMAAFNCAG | | | |

TABLE 4
abYsis database heavy chain sequences and
composition of residue at position 14.

| #Chothia | H14 | All |
|---|---|---|
| #Amino | Non-identical | Relative |
| Acid | Count | Frequency (%) |
| A | 1426 | 1.97 |
| C | 3 | 0.004 |
| D | 36 | 0.05 |
| E | 12 | 0.017 |
| F | 38 | 0.052 |
| G | 17 | 0.023 |
| H | 44 | 0.061 |
| I | 30 | 0.041 |
| K | 10 | 0.014 |
| L | 405 | 0.559 |
| M | 5 | 0.007 |
| N | 5 | 0.007 |
| P | 68803 | 95.03 |
| Q | 22 | 0.03 |
| R | 109 | 0.151 |
| S | 849 | 1.173 |
| T | 391 | 0.54 |
| V | 91 | 0.126 |
| W | 4 | 0.006 |
| Y | 12 | 0.017 |

TABLE 5
abYsis database heavy chain sequences and
composition of residue at position 20.

| #Chothia | H20 | All |
|---|---|---|
| #Amino | Non-identical | Relative |
| Acid | Count | Frequency (%) |
| A | 127 | 0.161 |
| C | 1 | 0.001 |
| D | 2 | 0.003 |
| E | 0 | 0 |
| F | 122 | 0.155 |
| G | 4 | 0.005 |
| H | 14 | 0.018 |
| I | 8658 | 11 |
| K | 8 | 0.01 |
| L | 57498 | 73.02 |
| M | 2272 | 2.886 |
| N | 0 | 0 |
| P | 69 | 0.088 |
| Q | 4 | 0.005 |
| R | 10 | 0.013 |
| S | 22 | 0.028 |
| T | 25 | 0.032 |
| V | 9813 | 12.46 |
| W | 2 | 0.003 |
| Y | 40 | 0.051 |

TABLE 6
abYsis database heavy chain sequences and
composition of residue at position 81.

| #Chothia | H81 | All |
|---|---|---|
| #Amino | Non-identical | Relative |
| Acid | Count | Frequency (%) |
| A | 124 | 0.147 |
| C | 2 | 0.002 |
| D | 2300 | 2.72 |
| E | 12957 | 15.322 |
| F | 14 | 0.017 |
| G | 136 | 0.161 |
| H | 1356 | 1.604 |
| I | 200 | 0.237 |
| K | 10439 | 12.345 |
| L | 412 | 0.487 |
| M | 494 | 0.584 |
| N | 913 | 1.08 |
| P | 24 | 0.028 |
| Q | 48687 | 57.576 |
| R | 1798 | 2.126 |
| S | 764 | 0.903 |
| T | 3704 | 4.38 |
| V | 97 | 0.115 |
| W | 5 | 0.006 |
| Y | 91 | 0.108 |

Two SHM were found in the light chain. A SHM was found at position 1 of LCFR and resulted into an Alanine at that position (FIG. 1B, Table 7). An Aspartic acid is the most frequently found residue at this position (frequency 37.355%) while an Ala at this position is relatively rare (frequency 6.099%) (Table 7, FIG. 2D). A SHM was also found at position 91 of the LCCDR, resulting in an Alanine residue at that position. A Proline at Position 91 is the most frequent residue found at this position (50.996%) whereas an Ala is relatively rare (frequency 2.332%) (Table 8, FIG. 2E).

TABLE 7
abYsis database light chain sequences and
composition of residue at position 1.

| #Chothia | L1 | All |
|---|---|---|
| #Amino | Non-identical | Relative |
| Acid | Count | Frequency (%) |
| A | 1474 | 6.099 |
| C | 3 | 0.012 |
| D | 9028 | 37.355 |
| E | 4268 | 17.66 |
| F | 31 | 0.128 |
| G | 135 | 0.559 |
| H | 54 | 0.223 |
| I | 39 | 0.161 |
| K | 58 | 0.24 |
| L | 43 | 0.178 |
| M | 42 | 0.174 |
| N | 345 | 1.428 |
| P | 42 | 0.174 |
| Q | 6544 | 27.077 |
| R | 39 | 0.161 |
| S | 1775 | 7.344 |
| T | 46 | 0.19 |
| V | 43 | 0.178 |
| W | 4 | 0.017 |
| Y | 135 | 0.559 |

TABLE 8
abYsis database light chain sequences and
composition of residue at position 91.

| #Chothia | L91 | All |
|---|---|---|
| #Amino | Non-identical | Relative |
| Acid | Count | Frequency (%) |
| A | 660 | 2.332 |
| C | 13 | 0.046 |
| D | 427 | 1.509 |
| E | 66 | 0.233 |
| F | 125 | 0.442 |
| G | 1644 | 5.809 |
| H | 457 | 1.615 |
| I | 285 | 1.007 |
| K | 166 | 0.587 |
| L | 3438 | 12.148 |
| M | 62 | 0.219 |
| N | 1191 | 4.208 |
| P | 14432 | 50.996 |
| Q | 119 | 0.42 |
| R | 319 | 1.127 |
| S | 3053 | 10.788 |
| T | 960 | 3.392 |
| V | 327 | 1.155 |
| W | 76 | 0.269 |
| Y | 450 | 1.59 |

To assess the potential impact of the SHM on binding to the target protein, binding epitopes were determined by HXMS. Four regions were identified as paratope defined regions by HDX-MS. The regions are distributed in three location in the heavy chain and one region in the light chain of the antibody outside of the framework where the somatic hyper mutations were observed (data not shown). Germlining these sites is therefore not expected to affect binding affinity and function.

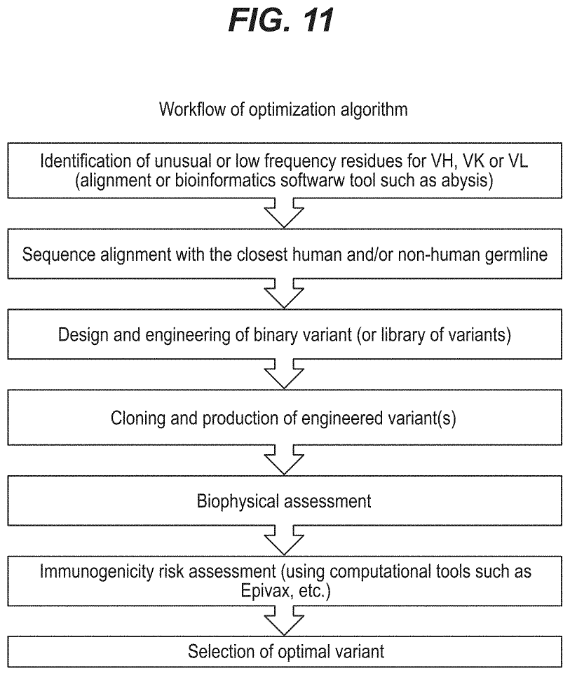

Germline variants with mutations either in the heavy chain SHM sites or the light chain SHM sites or in the heavy and light chain SHM sites combined were expressed and tested for both functional activity and intrinsic properties. A workflow as outlined in FIG. 11 was adopted to identify the SHM and engineer antibodies with improved molecular attributes. The library of binary variants constructed is described in Table 9. Functional and biophysical properties of each variants were tested as described below.

TABLE 9
Library of binary variants.

| B# | Heavy chain | HC mutations | | | Light chain | LC mutations | |
|---|---|---|---|---|---|---|---|
| TMEB746 | TMEH411 | R14P | P20L | H81Q | | | |
| TMEB747 | TMEH411 | R14P | P20L | | | | |
| TMEB748 | TMEH411 | R14P | | H81Q | | | |
| TMEB750 | TMEH411 | | P20L | H81Q | | | |
| TMEB751 | TMEH411 | | P20L | | | | |
| TMEB752 | | | | | TMEL127 | | A91P |
| TMEB756 | TMEH411 | R14P | P20L | H81Q | TMEL127 | | A91P |
| TMEB757 | TMEH411 | R14P | P20L | | TMEL127 | | A91P |
| TMEB758 | TMEH411 | R14P | H81Q | | TMEL127 | | A91P |
| TMEB759 | TMEH411 | R14P | | | TMEL127 | | A91P |
| TMEB760 | TMEH411 | P20L | H81Q | | TMEL127 | | A91P |
| TMEB761 | TMEH411 | P20L | | | TMEL127 | | A91P |
| TMEB762 | TMEH411 | R14P | P20L | H81Q | TMEL127 | A1D | A91P |
| TMEB763 | TMEH411 | R14P | P20L | | TMEL127 | A1D | A91P |
| TMEB764 | TMEH411 | R14P | H81Q | | TMEL127 | A1D | A91P |
| TMEB765 | TMEH411 | R14P | | | TMEL127 | A1D | A91P |
| TMEB766 | TMEH411 | P20L | H81Q | | TMEL127 | A1D | A91P |

### Biophysical Assessment

### Methods Used to Assess Biophysical Properties

### Differential Scanning Fluorimetry (DSF)

Thermal stability of antibody variants was determined by NanoDSF using an automated Prometheus instrument. Measurements were made by loading sample into 24 well capillary from a 384 well sample plate. Duplicate runs were performed for each sample. The thermal scans for a typical IgG sample span from 20° C. to 95° C. at a rate of 1.0° C./minute. Intrinsic tryptophan and tyrosine fluorescence at 330 nm and 350 nm emission wavelength, as well as the ratio F350 nm/F330 nm ratio were plotted against temperature to generate an unfolding curve. The back-reflection optics of the nanoDSF instrument emit near-UV light at a wavelength not absorbed by proteins. Aggregated proteins will scatter the light and non-scattered light will reach the detector. The reduction in back reflected light is a direct measurement of aggregation and is plotted as mAU (Attenuation Units) against temperature.

Thermal unfolding parameters (Tm and Tagg) of antibody variants were measured at 0.5 mg/mL in Phosphate Buffered Saline, pH 7.4.

Chemical denaturation experiments were carried out by incubating purified mAbs in different concentrations of GdnCl from OM to 6M overnight at room temperature. Intrinsic fluorescence was measured the next day using NanoDSF at 25° C. The F350 nm/F330 nm ratio was plotted at each concentration of GdnCl to generate an unfolding curve that was fitted either by a two-state or three-state unfolding equations to obtain the free energy of unfolding (ΔGu) and concentration of denaturant at which 50% of molecules exist as unfolded (C_{1/2} aka C₅₀).

### Differential Scanning Calorimetry (DSC)

Thermal stability was characterized by capillary VP-DSC microcalorimeter (Microcal Inc. Northampton, Mass.). Temperature scans were performed from 25 to 120° C. at a protein concentration of 1.0 mg/mL and a scan rate of 1° C./min. A buffer reference scan was subtracted from the protein scan and the concentration of protein was normalized prior to thermodynamic analysis. The DSC curve was fitted using non-two-state model to obtain the enthalpy and apparent transition temperature (Tm) values.

### Nonspecific Binding

Nonspecific binding of the lead molecule to unrelated surfaces was determined by biosensor technology (BIAcore 8K). Antibody variants at a concentration of 104 were passed over SPR surfaces coated with unrelated proteins. An antibody displaying significant binding to irrelevant surfaces is predicted to have poor in-vivo properties and manufacturing challenges. Irrelevant surfaces include negatively and positively charged proteins, hydrophobic proteins, and human IgG.

### Analytical Ultra Centrifugation

A Beckman Optima AUC instrument was used to measure quantitative size distribution of monomers and other higher order aggregates of proteins in solution by analytical ultra-centrifugation. Samples were loaded into centrifuge cells equipped with 1.2 cm Beckman centerpieces (rated to 50K rpm) and quartz windows. The cells were assembled and torqued to 130 lbs. The centrifuge cells were placed into an An-50 (8 hole) or An-60 (4 hole) rotor and placed within the AUC chamber. The temperature of the AUC instrument was set to 20.5° C. for at least one hour before initiating the run. Runs were performed at 40K rpm 250 scan counts (250 scans), 90 seconds frequency of scan collection, 10 μM data resolution and at a wavelength of 280 nm. The data were analyzed using the direct boundary fitting software SEDANAL.

### Short Term Stability (4° C., 40° C.)

Concentrated mAbs were tested by analytical size exclusion chromatography (SEC-HPLC) to measure the percentage of monomer. MAbs were then incubated at 4° C. and 40° C. for 4 weeks. Aliquots were drawn at regular intervals and integrity was checked by SEC-HPLC.

### Molecular Modeling

Molecular homology models were generated using the MOE modeling software (CCG, Montreal) using its standard antibody modeling protocols. Germlining mutations were identified and highlighted in the stick representation. Molecular figures were generated in the computer graphics program PyMol.

### Results of Biophysical Evaluation

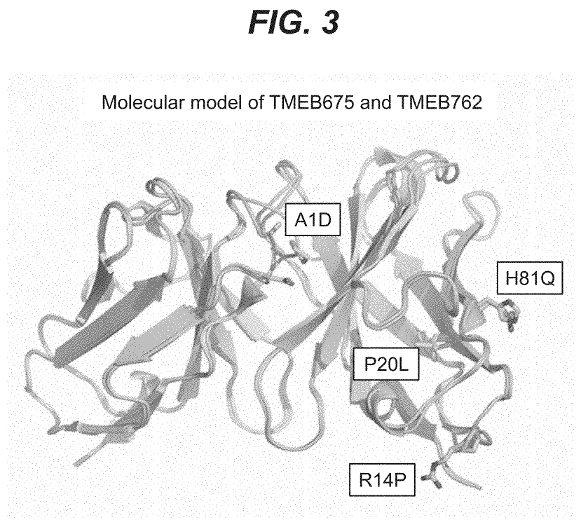

Re-engineering of the SHM residues led to the discovery of optimized variants with better biophysical attributes. Of the 11 variants tested, TMEB762 containing the three heavy chain reengineered germline mutations, R14P, P20L and H81Q, and two light chain germline mutations, MD and A91P, had the most optimal biophysical properties. Residue numbering is according to Kabat. To better understand the effect of the SHM and the structural basis for thermal stabilization, the five germline mutations were mapped onto the molecular models for both Fv's, (MOE, CCG, Montreal) (FIG. 3). Of the five germline mutations, MD and H82H are surfaced exposed and have therefore likely little contribution to domain stability. The VH R14P may have impact on the structure and may therefore have a slight effect on domain stability. According molecular modeling, he VH P20L and VL A95P mutations are likely two major structural determinants. P20L in located in the middle of a β-strand with its side chain buried in the VH core. Proline is not a favorable residue in a typical β-strand structure. A Leu at this position would restore a favorable residue and a Leucine sidechain would pack well in the core. Amino acid residues at position 95 in this class of VL are typically in the cis conformation which maximizes stability. The effects of non-Pro mutations at this position upon stability and structure was previously reported (Luo, J. et al. Coevolution of antibody stability and Vkappa CDR-L3 canonical structure. J Mol Biol 402, 708-719 (2010)). An Ala at this position is likely to distort the local canonical structure or be forced into energetically unfavorable non-Pro cis peptide bond. Both would have negative consequences for stability. Overall, the rationale for the germline mutations are well supported.

### Biophysical Characterization

### Analytical Ultra Centrifugation (AUC)

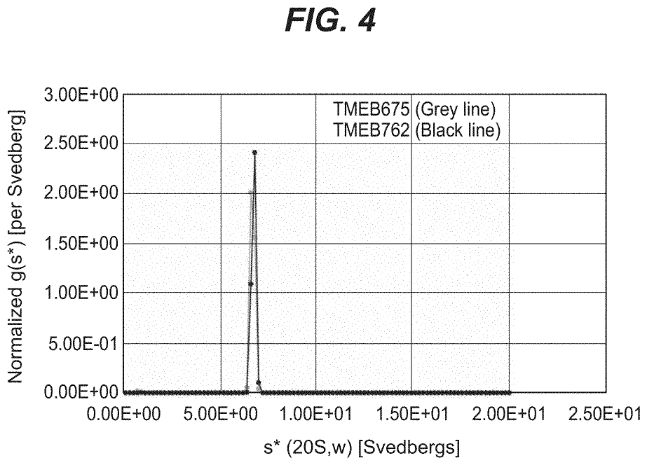

Presence of minute quantities of process and product related impurities pose major threat to safety and raise immunogenicity related risks. Conducting biophysical characterization on a high-quality molecule is essential to truly determine its intrinsic properties. AUC is a powerful technique to measure the quantitative size distribution of monomers and other higher order aggregates of proteins in solution (Berkowitz, S. A. Role of analytical ultracentrifugation in assessing the aggregation of protein biopharmaceuticals. AAPS J 8, E590-605 (2006)). Particularly, Sedimentation Velocity (SV) based analysis uniquely measures hydrodynamic size and shape of proteins in any buffer in an unbiased way. Analytical Ultracentrifugation Sedimentation Velocity (AUC-SV) runs of both TMEB762 (black line) and TMEB675 (grey line) are shown in FIG. 4. Based on SV-AUC analysis, both TMEB675 and TMEB762 exhibited >95% monomer after purification and are therefore good starting material for further biophysical characterization.

### Thermal Stability

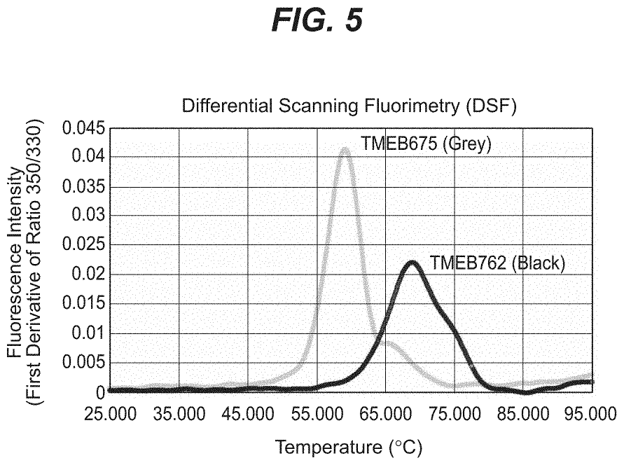

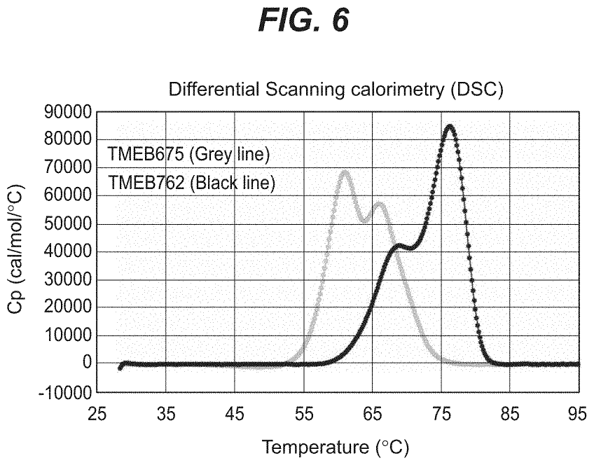

Both conformational and colloidal stability are well demonstrated manufacturability parameters that predict stability, shelf-life and successful drug development. Simultaneous assessment of both parameters is a very powerful approach for long term stability determination. Temperature is one of the widely used denaturation methods to dissect the structural stability of a molecule. Tryptophan based fluorescence emission was used for monitoring thermal unfolding of both mAbs in PBS using Prometheus NT.48 instrument. High fluorescence sensitivity detection enables the monitoring of mAb conformational changes due to different subdomain unfolding. At the same time, fluorescence detection can detect changes in colloidal stability by monitoring temperature induced aggregation using back-reflection light intensity technology. Likewise, differential scanning calorimetry is the industry gold standard thermal melting tool for determining domain-based stability at higher temperatures. FIG. 5 provides the thermal unfolding profile of TMEB675 and TMEB762 as determined by Nano DSF. The data shows that TMEB762 is more stable. Unfolding of TMEB762 starts at a higher temperature than for TMEB675, around 59 C with Fab unfolding occurring close to 75° C. (Table 10). The antibody is very stable and shows no sign of aggregation (Tagg) below that temperature. FIG. 6 shows the thermal unfolding profile of TMEB675 and TMEB762 as determined by DSC.

TABLE 10
Thermal Stability Parameters determined from DSC (on-set
of unfolding and Fab domain unfolding Tm) and DSF (Tagg).

| | Ton-set - DSC | | Tagg - DSF |
|---|---|---|---|
| mAb | (° C.) | Tm (Fab) - DSC | (° C.) |
| TMEB675 | 52.9 | 61.8 | 61.5 |
| TMEB762 | 59.3 | 75.5 | 75.7 |

### Free Energy of Unfolding

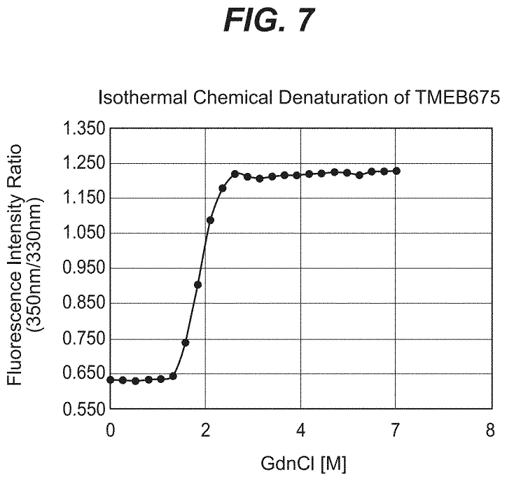

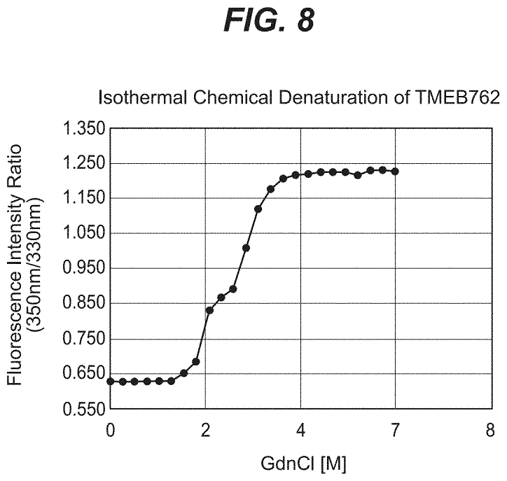

Undoubtedly thermal denaturation experiments are one of the commonest stability determination tools available as high throughput for rank ordering molecules early on. However, the existing challenge is to accurately calculate the intrinsic stability at lower temperatures based on higher temperature data. The calculation is prone to error since thermal melting is often irreversible due to aggregation which precludes extrapolation of reliable stability parameters at lower temperatures (Freire, E., Schon, A., Hutchins, B. M. & Brown, R. K. Chemical denaturation as a tool in the formulation optimization of biologics. Drug Discov Today 18, 1007-1013 (2013). In addition, stability of lead candidates is often only measured at 25° C. or 37° C. Isothermal Chemical Denaturation (ICD) at a single temperature is a proven reliable thermodynamic analysis to provide the intrinsic stability of a protein in any solvent (Svilenov, H., Markoj a, U. & Winter, G. Isothermal chemical denaturation as a complementary tool to overcome limitations of thermal differential scanning fluorimetry in predicting physical stability of protein formulations. Eur J Pharm Biopharm 125, 106-113 (2018)). In an ICD experiment, mAb are incubated at a given concentration in increasing concentrations of a denaturing chemical for 12-16 hours minimum, before measuring conformational change. The change in F350/F330 fluorescence ratio is used to determine the fraction of unfolded protein at each measured concentration of denaturing chemical. The Gibbs free energy of unfolding (ΔGu) calculated from the fitting curves is an indicator of intrinsic conformational stability of the mAb at a particular temperature. Another important parameter from this fitting is c50, which represents the concentration of the denaturant at which 50% of the antibody is unfolded. FIG. 7 and FIG. 8 provide the ICD unfolding curves of TMEB675 and TMEB762 measured at 25° C. It is interesting to note that TMEB675 exhibits a single transition with a ΔGu of 24.3 kJ/mol while TMEB762 shows three state of unfolding, typical of a well behaved mAb, with a first transition ΔGu of 63.5 kJ/mol and a second transition of ΔGu of 37.3 kJ/mol. The approximate threefold increase in the free energy of unfolding of the first transition of TMEB762 clearly demonstrates that TMEB762 is intrinsically more stable than TMEB675 likely because of its germline optimized FAB domain (Table 11).

TABLE 11
Intrinsic Stability Parameters from ICD experiments.
ΔGu1, ΔGu2, C₅₀ are the calculated parameters
from 2-state and 3-state fitting of GdnCl induced
denaturation curves generated in nano DSF experiment.

| | ΔGu1 | C₅₀ | ΔGu2 | C₅₀ |
|---|---|---|---|---|
| mAb | (kJ/mol) | [M] | (kJ/mol) | [M] |
| TMEB675 | 24.3 | 1.8 | NA | NA |
| TMEB762 | 63.5 | 1.9 | 37.2 | 2.9 |

### Storage Stability Assessment

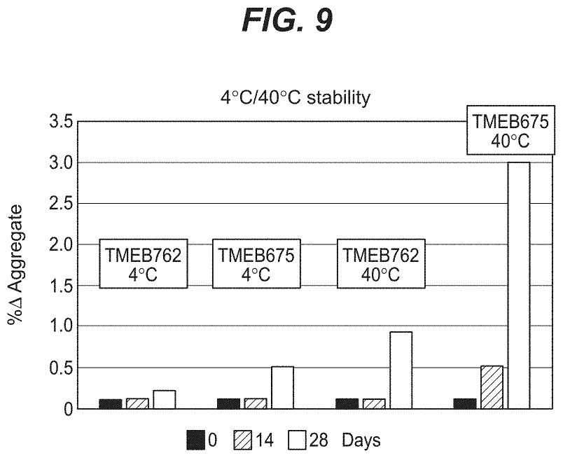

Accelerated thermal stress is an industry wide used forced degradation assay to generate enough degradation product and understand the degradation mechanism of an antibody in a shortened timeline. It is used as a direct prediction for long-term shelf stability. Both long term storage (4° C.) and accelerated storage (40° C.) were studied for TMEB675 and TMEB762 for a month in PBS by monitoring their degradation by analytical size exclusion chromatography (aSEC). aSEC chromatograms (data not shown), showed that the antibodies degraded overtime through aggregation and not fragmentation. Change in aggregate levels between time zero, 2 weeks and 4 weeks were plotted for both mAbs (FIG. 9). TMEB762 had <0.3% aggregates at 4° C. and <1% aggregates at accelerated storage at 40° C. for a month. TMEB675, however showed 0.5% and 3% aggregation increase after a month at 4° C. and 40° C. respectively. As consistent with various literature, higher thermal stability correlates with lower propensity for aggregation (Brader, M. L. et al. Examination of thermal unfolding and aggregation profiles of a series of developable therapeutic monoclonal antibodies. Mol Pharm 12, 1005-1017 (2015); He, F. et al. Detection of IgG aggregation by a high throughput method based on extrinsic fluorescence. J Pharm Sci 99, 2598-2608 (2010)).

### Non-Specific Binding

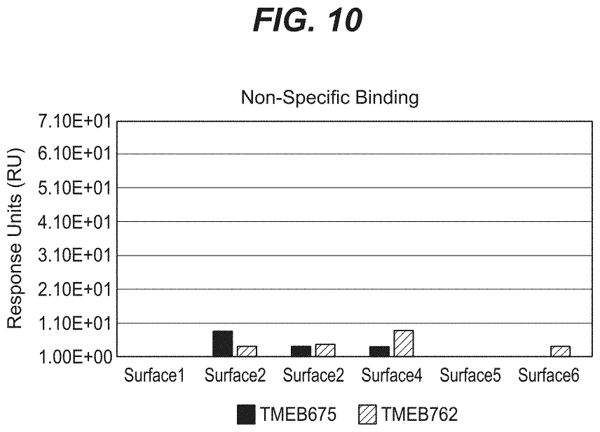

Sequence optimization of lead candidates may sometimes lead to unexpected modifications of their physical properties such as hydrophobicity, charge heterogeneity, folding, solubility, and solvent accessibility. Modification of these intrinsic properties will have major impact on developability and pharmacokinetic behavior. Faster clearance of mAbs can be attributed to nonspecific interactions with other irrelevant proteins in vivo. These simple physical properties can be measured by nonspecific binding assays (Dostalek, M., Prueksaritanont, T. & Kelley, R. F. Pharmacokinetic de-risking tools for selection of monoclonal antibody lead candidates. MAbs 9, 756-766 (2017)). Here we used Surface Plasmon Resonance (SPR) based non-specific binding assay to determine non-specific binding properties of both TMEB675 and TMEB762 to hydrophobic, charged, and IgG surfaces. Based on experimental data collected for many early and late stage candidates including marketed antibodies, we have come up with the criteria (not revealed here) for relative binding response for candidates that are tested for non-specific binding. Proper control antibodies (positive and negative) are run in every single experiment for validation. Relative Response Units of TMEB675 and TMEB762 was plotted against the binding to different surfaces. Binding response to control dextran surface flow cell was subtracted from each data set. TMEB762 and TMEB675 show no non-specific binding to any of the charged surfaces tested even at 1 μM concentration (FIG. 10). Non-specific binding to any irrelevant surfaces could have been a significant challenge for developing these mAbs with a potential concern about in-vivo behavior.

The biophysical assessment showed that germlining the framework residues has safely and favorably enhanced the thermal stability of TMEB762 and lowered the aggregation propensity without significantly altering the conformation of the antibody.

### Immunogenicity Risk Assessment

TMEB762 showed reduced risk of immunogenicity compared to TMEB675 as indicated by the improvement in % Human germline sequence identity. In addition, the insilico immunogenicity risk assessment scores drastically improved as well (Table 12). EpiVax screens for immunogenicity and relies on a set of immunoinformatic tools to predict the immunogenicity of peptides and proteins.

TABLE 12
Immunogenicity risk assessment of TMEB675
and engineered variant TMEB762.

Immunogenicity risk assessment

| | Epivax score | % Human |
|---|---|---|
| | (Treg adjusted) | (framework) |
| TMEB675 (parental) | VH = 0.96 | VH = 95% |
| | Vk = −19.3 | Vk = 100% |
| TMEB762 (Engineered) | VH = −44.9 | VH = 99% |
| | Vk = −33.6 | Vk = 100% |

### Binding Affinity

The binding affinity of TMEB762 was measured and compared to TMEB675 and is summarized in Table 13.

TABLE 13
Parameters from Affinity measurement done by SPR are provided in
this table. Association constant kₐ (M⁻¹ s⁻¹), dissociation constant k_{d}
((s⁻¹) and equilibrium constant K_{D} (M) are included.

| | kₐ | k_{d} | K_{D} |
|---|---|---|---|
| mAb | (M⁻¹ s⁻¹) | (s⁻¹) | (pM) |
| TMEB675 | 6.24 × 10⁴ | 3.16 × 10⁻⁵ | 25.7 |
| TMEB762 | 1.00 × 10⁵ | 2.27 × 10⁻⁵ | 23.7 |

### Conclusion

Out of 11 variants tested, TMEB762 had the most desirable functional and biophysical properties. Germlining of TMEB675 led to a more conformationally stable TMEB762 that has a very low propensity to aggregate (<1%) and aligns with the quality attributes for FDA/EMA approved and clinical stage mAb candidates.

### Example 2

The workflow described in Example 1 was applied to optimize other antibodies of different structure and function. Example 2 describes the optimization of an anti-prostate target antibody, PSMW56, through germlining of SHM sites identified in the framework of the antibody.

### Discovery, Engineering and Germline Optimization

The monoclonal antibodies (PSMW56) were discovered by immunizing OmniRat with the recombinant human PSMA proteins in the OmniRat® transgenic platform. Following a 89-day immunization regimen, lymph nodes from the rats were harvested and used to generate hybridomas. Hybridoma supernatants were screened for binding to recombinant antigens by ELISA. Based on the screening results, several hybridoma clones were sequenced, expressed and characterized for functionality. Variant PSMW56 showed desirable recombinant protein affinity (Table 14) and was selected for further studies. While the antibody met the functional criteria characteristics of a high affinity antibody, it showed poor thermal stability.

TABLE 14
Parameters from Affinity measurement of equilibrium
constant K_{D} (M) as determined by SPR.

| | K_{D} |
|---|---|
| mAb | (nM) |
| PSMW56 | 8.6 |

### Engineering of Anti-PSMA Antibody

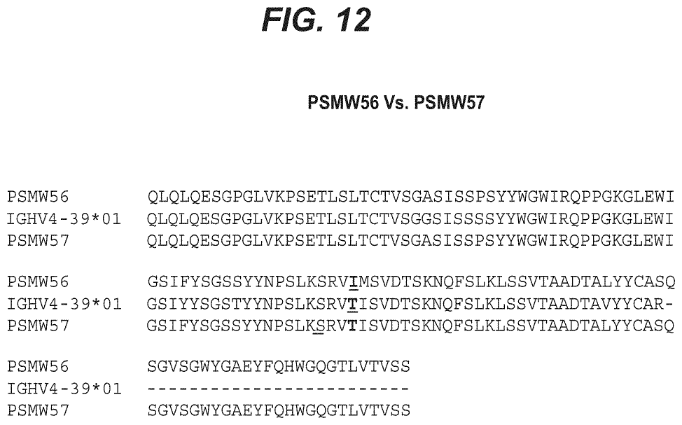

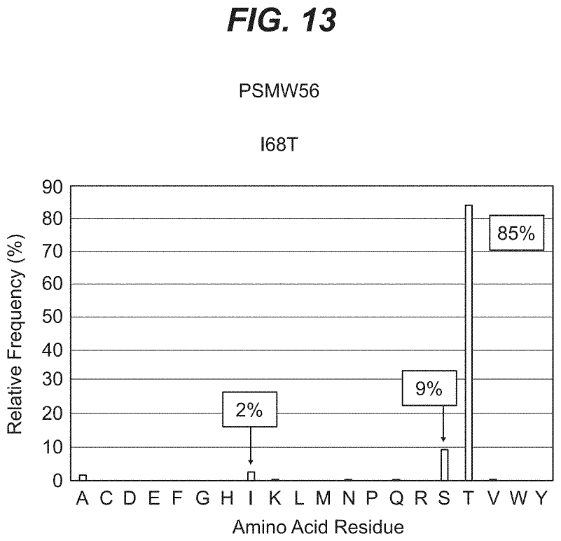

The sequence alignment of the variable heavy chain region of PSMW56 with human germline sequences for VH using the abYsis portal indicated several somatic hypermutations (SHM) within the framework region. One somatic hypermutations were observed in VH (Ile68) (FIG. 12 and FIG. 13). Threonine is the frequent amino acid residues found at position 68. The SHM of Ile found at position 68 is low frequency (3%) compared to the most frequently found Thr residue at this position (Thr residue frequency 85%) Table 15 shows abYsis database heavy chain sequences and composition of residue at position 68. An engineered variant PSMW57 was generated by replacing Ile68 with Thr on the parental clone PSMW56.

TABLE 15
abYsis database heavy chain sequences and composition
of residue at position 68 for anti-PSMA.

| #Chothia | H68 | All |
|---|---|---|
| #Amino | Non-identical | Relative |
| Acid | Count | Frequency (%) |
| A | 1260 | 1 |
| C | 1 | <1 |
| D | 42 | <1 |
| E | 153 | <1 |
| F | 100 | <1 |
| G | 34 | <1 |
| H | 25 | <1 |
| I | 2219 | 3 |
| K | 244 | <1 |
| L | 48 | <1 |
| M | 47 | <1 |
| N | 252 | <1 |
| P | 30 | <1 |
| Q | 175 | <1 |
| R | 108 | <1 |
| S | 8215 | 9 |
| T | 74302 | 85 |
| V | 468 | <1 |
| W | 0 | <1 |
| Y | 85 | <1 |

### Biophysical Assessment of the Anti-PSMA Antibody Variant—Thermal Stability

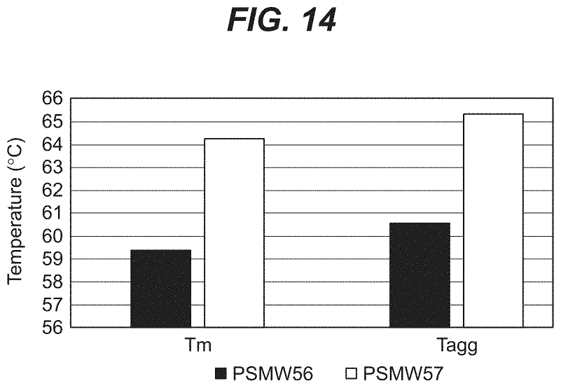

A germline variant (PSMW57) with the heavy chain I68T mutation was expressed and tested for thermal stability. The engineered anti-PSMA variant (PSMW57) showed significant increase in thermal stability (both Tm and Tagg) when compared to PSMW56 as shown in FIG. 14 demonstrating that the workflow described in FIG. 11 is applicable to other antibodies.

### Example 3

To further demonstrate that the workflow of FIG. 11 is broadly applicable to other antibodies, the workflow was applied to optimize the anti-prostate cancer antibody, DL3B355. The example below describes the optimization of DL3B355, through germlining of SHM sites identified in the framework of the antibody.

### Discovery, Engineering and Germline Optimization

Anti-DLL3 monoclonal antibodies were discovered by immunizing AlivamAb mice, a transgenic fully human antibody platforms that produces a diverse repertoire of antibodies with human idiotypes with the recombinant human DLL3. Hybridoma supernatants were screened for binding to recombinant antigens by ELISA.

Based on the screening results, several hybridoma clones were sequenced, expressed and characterized for functionality. DL3B355 showed desirable recombinant protein affinity (Table 16) and was selected for further studies.

TABLE 16
Parameters from Affinity measurement of equilibrium
constant K_{D} (M) were determined by SPR.

| | K_{D} |
|---|---|
| mAb | (nM) |
| DL3B355 | 0.166 |

As described in Example 1, the AbYsis tool was used to search for “unusual residues” within the antibody heavy and light chain sequence. Unusual residues defined by a 1% threshold in the database of antibody sequences offers hints about the critical functions of certain positions.

Engineering of Anti-DLL3 Antibody

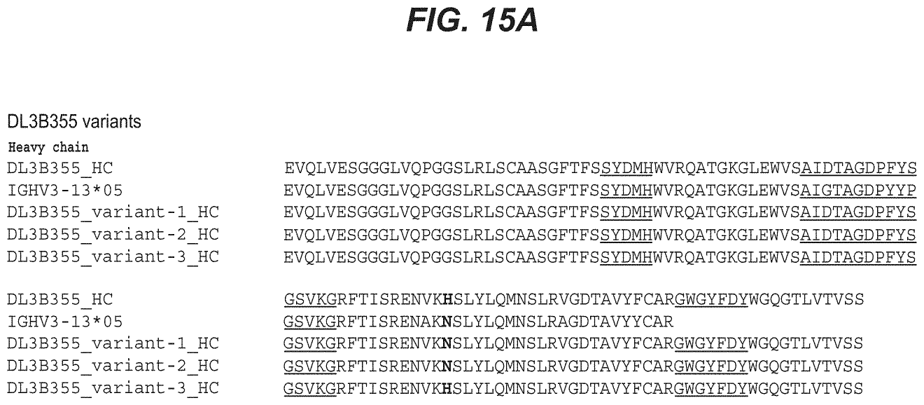

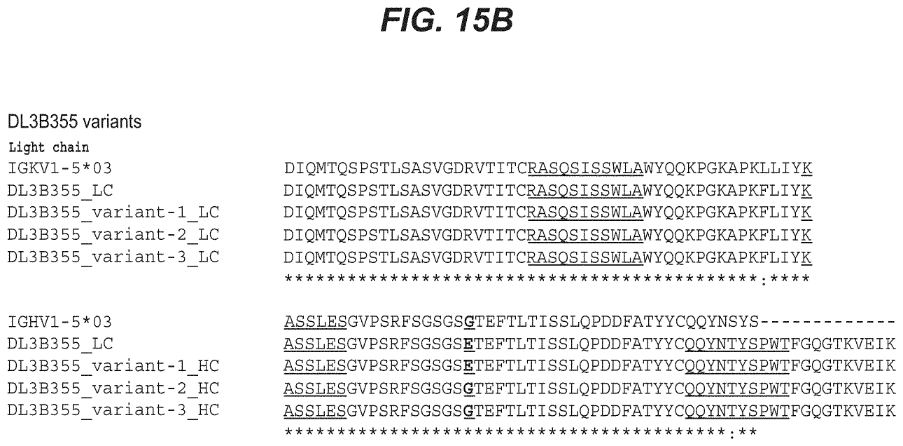

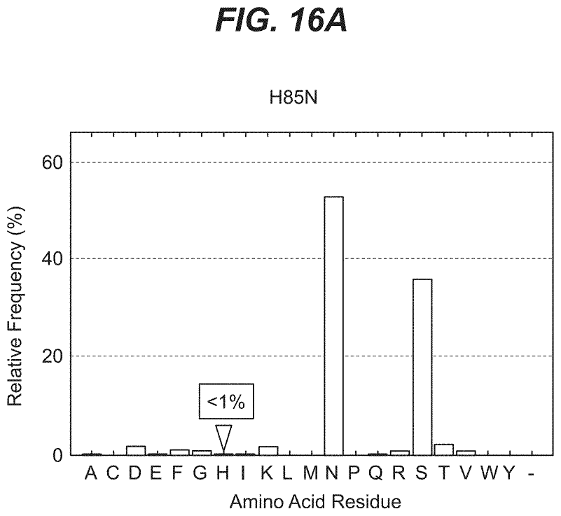

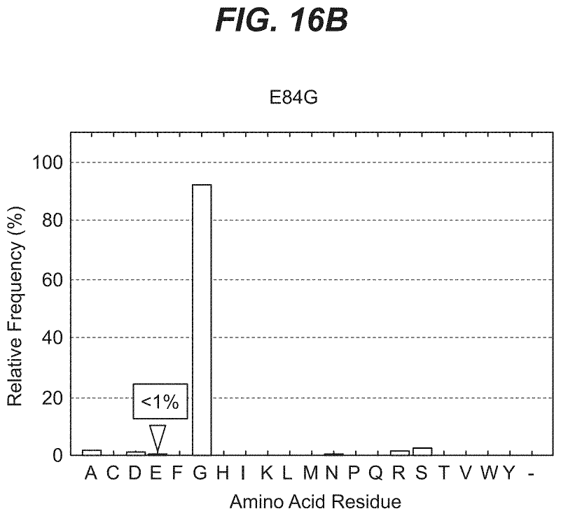

The sequence alignment of the variable heavy chain and light region of DL3B355 with human germline sequences for VH and VL using the abYsis portal indicated several somatic hypermutations (SHM) within the framework region. One somatic hypermutations were observed in VH (His85) (FIG. 15A and FIG. 16B). In the heavy chain, asparagine is the frequent amino acid residues found at position 85. The SHM of His found at position 85 is rare frequency (<1%) compared to the most frequently found Asn residue at this position (Asn residue frequency 52%, Table 17). One somatic hypermutations were observed in VL (Glu84) (FIG. 15B and FIG. 16B). In the heavy chain, Glycine is the frequent amino acid residues found at position 84. The SHM of Glu found at position 84 is rare frequency (<1%) compared to the most frequently found Gly residue at this position (Gly residue frequency 93%, Table 18). Three engineered variants of DL3B355 were generated as depicted in Table 19.

TABLE 17
abYsis database heavy chain sequences and composition
of residue at position 85 for anti-DLL3.

| #Chothia | H85 | All |
|---|---|---|
| #Amino | Non-identical | Relative |
| Acid | Count | Frequency (%) |
| A | 270 | 0.319 |
| C | 16 | 0.019 |
| D | 1600 | 1.892 |
| E | 435 | 0.514 |
| F | 872 | 1.031 |
| G | 710 | 0.84 |
| H | 175 | 0.207 |
| I | 152 | 0.18 |
| K | 1461 | 1.728 |
| L | 54 | 0.064 |
| M | 43 | 0.051 |
| N | 44650 | 52.801 |
| P | 15 | 0.018 |
| Q | 323 | 0.382 |
| R | 713 | 0.843 |
| S | 30462 | 36.023 |
| T | 1925 | 2.276 |
| V | 517 | 0.611 |
| W | 5 | 0.006 |
| Y | 112 | 0.132 |

TABLE 18
abYsis database light chain sequences and composition
of residue at position 84 for anti-DLL3.

| #Chothia | L84 | All |
|---|---|---|
| #Amino | Non-identical | Relative |
| Acid | Count | Frequency (%) |
| A | 612 | 2 |
| C | 3 | <1 |
| D | 318 | <1 |
| E | 266 | <1 |
| F | 7 | <1 |
| G | 33765 | 93 |
| H | 43 | <1 |
| I | 1 | <1 |
| K | 17 | <1 |
| L | 5 | <1 |
| M | 6 | <1 |
| N | 179 | <1 |
| P | 2 | <1 |
| Q | 14 | <1 |
| R | 346 | <1 |
| S | 700 | 2 |
| T | 59 | <1 |
| V | 58 | <1 |
| W | 10 | <1 |
| Y | 37 | <1 |

TABLE 19
Library of engineered variants for anti-DLL3.

| | | Heavy | Light |
|---|---|---|---|
| # | Name | chain | chain |
| 1 | DL3B355-variant1 | H85N | — |
| 2 | DL3B355-variant2 | H85N | E84G |
| 3 | DL3B355-variant3 | — | E84G |

### Biophysical Assessment of the DLL3 Antibody Variants—Thermal Stability

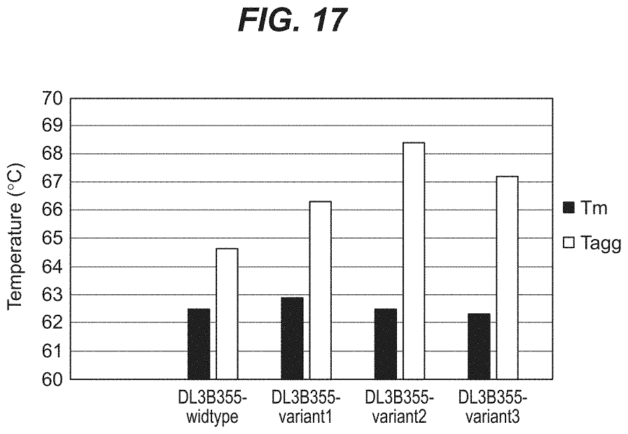

Thermal Stability Parameters were measured for each engineered anti-DLL3 variant to determine whether the germline mutations had a positive effect on biophysical attributes. On-set of unfolding and Fab domain unfolding Tm were measured by DSF and DSC. The engineered anti-DLL3 variants showed significant increase in thermal stability (both Tm and Tagg) as shown in FIG. 17.

All publications, including but not limited to patents and patent applications, cited in this specification are herein incorporated by reference as though fully set forth.

1. A method for optimizing an antibody comprising a variable heavy chain (VH) and/or a variable light chain (VL), the method comprising:
a) Identifying an antibody for optimizing;
b) Identifying one or more unusual or low frequency residues in said antibody VH and/or VL;
c) Aligning said antibody VH and/or VL sequences with the closest human or non-human germline sequences;
d) Identifying one or more somatic hypermutation sites in said antibody VH, VL, or both;
e) Identifying one or more germline residues typically observed at the site of said somatic hypermutation sites;
f) Designing and engineering variants or a library of variants containing said germline residues at the site of said somatic hypermutation sites;
g) Assessing properties of said variants or library of variants; and
h) Selecting one or more optimized variants,
wherein said one or more optimized variants has improved biophysical properties, decreased risk of immunogenicity, or both.

2. The method of claim 1, wherein the identification of unusual or low frequency residues is done by a computer-based software.

3. The method of claim 2, wherein the computer-based software is abYsis.

4. The method of claim 1, wherein the said unusual or low frequency residue is in the antibody VH.

5. The method of claim 1, wherein the said unusual or low frequency residue is in the antibody VL.

6. The method of claim 1, wherein the said unusual or low frequency residue is in the antibody VH and VL.

7. The method of claim 1, wherein said lead antibody contains somatic hypermutations in one or more of the framework regions (FRs) and/or complementarity determining regions (CDRs).

8. The method of claim 1, wherein the engineered variants are made in the human framework region, CDR1, CDR2 or CDR3 of the antibody.

9. The method of claim 1, wherein aligning said antibody VH and/or VL sequences is with the closest human germline sequences.

10. The method of claim 1, wherein the method further comprises cloning and producing said variants or library of variants.

11. The method of claim 1, wherein the assessment of said variants or library of variants is a biophysical assessment.

12. The method of claim 11, wherein the biophysical assessment is analytical ultracentrifugation, thermal stability, free energy of unfolding, analytical size exclusion, storage stability, and/or non-specific binding.

13. The method of claim 12, wherein analytical ultracentrifugation assessment further comprises comparing the Analytical Ultracentrifugation Sedimentation Velocity (AUC-SV) of said engineered variants, to the AUC-SV of said lead antibody.

14. The method of claim 12, wherein thermal stability assessment further comprises comparing the Tm of the thermal unfolding curve of each said engineered variant, to the Tm of the thermal unfolding curve of said lead antibody.

15. The method of claim 12, wherein thermal stability assessment further comprises comparing the Tagg of each said engineered variant, to the Tagg of said lead antibody.

16. The method of claim 12, wherein the free energy of unfolding further comprises comparing the ΔGu1, ΔGu2, or C₅₀ of each said engineered variant, to the ΔGu1, ΔGu2, or C₅₀ of said lead antibody.

17. The method of claim 12, wherein the storage stability of said engineered variant is measured at 4° C. or 40° C. at 2 weeks and 4 weeks and compared to the storage stability of said lead antibody.

18. The method of claim 1, wherein said optimal variant has increased monomer content, increased Tm, increased Tagg, increased ΔGu1, increased ΔGu2, increased C₅₀ values or reduced aggregation.

19. The method of claim 18, wherein said optimal variant has an increase in monomer content of 2% or more when compared to said lead antibody.

20. The method of claim 18, wherein said optimal variant has Tm increase of 1° C. or more when compared to said lead antibody.

21. The method of claim 18, wherein said optimal variant has Tagg increase of 1° C. or more, when compared to said lead antibody.

22. The method of claim 18, wherein said optimal variant has a free energy of unfolding ΔGu1 or ΔGu2 increase of 4 kJ/mol or more when compared to said lead antibody.

23. The method of claim 18, wherein said optimal variant has a C₅₀ increase of 0.1 M or more when compared to said lead antibody.

24. The method of claim 18, wherein said optimal variant has a decreased aggregation content of 1% or more when compared to said lead antibody.

25. The method of claim 1, wherein the assessment of said variants or library of variants is an immunogenicity risk assessment.

26. The method of claim 25, wherein the immunogenicity risk assessment is measured in silico.

27. The method of claim 26, wherein the immunogenicity risk assessment in silico is measured by Epivax score.

28. The method of claim 27 wherein said optimal variant has an Epivax score equal or lower when compared to said lead molecule.

29. The method as in any of the preceding claims, in which the antibody is an antibody, or antigen-binding fragment of an antibody.

30. A product produced by the method of any of the preceding claims.
